SEQUENCE LISTING (5 sequences)

SEQ ID NO: 1 - protein, 419 aa, SARS-CoV-2
  MISC_FEATURE (1)..(419): nucleoprotein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 1273 aa, SARS-CoV-2
  MISC_FEATURE (1)..(1273): spike protein
  MISC_FEATURE (13)..(685): SARS-CoV-2 S1 Protein
  MISC_FEATURE (319)..(541): RBD domain
  MISC_FEATURE (686)..(1273): SARS-CoV-2 S2 Protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 75 aa, SARS-CoV-2
  MISC_FEATURE (1)..(75): envelope protein
MYSFVSEETGTLIVNSVLLFLAFVVFLLVTLAILTALRLCAYCCNIVNVSLVKPSFYVYSRVKNLNSSRVPDLLV

SEQ ID NO: 4 - protein, 222 aa, SARS-CoV-2
  MISC_FEATURE (1)..(222): membrane protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 5 - protein, 1273 aa, SARS-CoV-2
  MISC_FEATURE (1)..(1273): spike protein
  MISC_FEATURE (13)..(685): SARS-CoV-2 S1 Protein
  MISC_FEATURE (319)..(541): RBD domain
  MISC_FEATURE (342)..(342): Xaa = any amino acid
  MISC_FEATURE (354)..(354): Xaa = any amino acid
  MISC_FEATURE (367)..(367): Xaa = any amino acid
  MISC_FEATURE (435)..(435): Xaa = any amino acid
  MISC_FEATURE (458)..(458): Xaa = any amino acid
  MISC_FEATURE (483)..(483): Xaa = any amino acid
  MISC_FEATURE (683)..(683): Xaa = any amino acid
  MISC_FEATURE (685)..(685): Xaa = any amino acid
  MISC_FEATURE (686)..(1273): SARS-CoV-2 S2 Protein
  MISC_FEATURE (986)..(987): Xaa = any amino acid
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVXNATRFASVYAWXRKRISNCVADYSXLYNSASFSTFKCYGVSPTKLNDLCFTNVYADSFVIRGDEVRQIAPGQTGKIADYNYKLPDDFTGCVIXWNSNNLDSKVGGNYNYLYRLFRXSNLKPFERDISTEIYQAGSTPCNGXEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRXAXSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDXXEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2021/0349105 A1
(43) Pub. Date: Nov. 11, 2021

(54) SARS-COV-2 IMMUNOASSAY AND MATERIALS THEREFOR

(71) Applicant: BIO-RAD LABORATORIES, INC., Hercules, CA (US)

(72) Inventors: ROGER P. WALKER, BENICIA, CA (US); HEIDI SCHOLZ, VACAVILLE, CA (US)

(21) Appl. No.: 17/314,376

(22) Filed: May 7, 2021

Related U.S. Application Data
(60) Provisional application No. 63/021,628, filed on May 7, 2020.
(60) Provisional application No. 63/032,541, filed on May 30, 2020.
(60) Provisional application No. 63/114,675, filed on Nov. 17, 2020.

(51) Int. Cl. G01N 33/68; G01N 33/569; G01N 33/533

(52) U.S. Cl. CPC G01N 33/6854; G01N 33/533; G01N 33/56983

(57) ABSTRACT

The present disclosure relates to the development of novel immunoassays for the detection of SARS-CoV-2 antigens and/or SARS-CoV-2 specific antibodies and, optionally, one or more cytokine in patient samples.

### CROSS-REFERENCE TO RELATED APPLICATIONS

This application claims the benefit of U.S. Provisional Application Ser. No. 63/021,628, filed May 7, 2020, Ser. No. 63/032,541, filed May 30, 2020 and Serial No. 63/114,675, filed Nov. 17, 2020, the disclosures of which are hereby incorporated by reference in their entirety, including all figures, tables and amino acid or nucleic acid sequences.

The Sequence Listing for this application is labeled “Seq-List.txt” which was created on May 5, 2021 and is 30 KB. The entire content of the sequence listing is incorporated herein by reference in its entirety.

### BACKGROUND

SARS-CoV-2, a coronavirus, is the causative viral agent of the disease COVID-19 which is a highly infectious human respiratory infection that threatens global public health. As of April 2021, this virus was known to have infected at least 152 million people worldwide with about 3.19 million known deaths.

Coronavirus (CoV) is an enveloped virus that contains a single-stranded positive-sense RNA. SARS-CoV-2, formerly known as 2019-nCoV, is a newly emerging coronavirus that mainly affects the respiratory tract that can lead to Severe Acute Respiratory Syndrome (SARS). The underlying disease caused by this virus is named COVID-19. Coronaviruses have been responsible for several outbreaks in the world during the two last decades. In 2003 and 2014, coronaviruses caused outbreaks mainly in Asia (SARS-CoV) and in the Middle East (MERS-CoV), respectively. Before the emergence of the new SARS-CoV-2, six coronaviruses were known to affect humans (SARS-CoV, MERS-CoV and four other coronaviruses that cause mild upper and lower respiratory syndromes).

SARS-CoV-2 was first identified in December 2019, in Wuhan City, Hubei Province, China, after several patients developed severe pneumonia similar to that caused by SARS-CoV. The virus has since rapidly spread around the globe and in March 2020, WHO officially announced COVID-19 as a pandemic. Person to-person transmission of the virus resulted in quick spreading of COVID-19 and the high number of patients requiring intensive care resulted in the establishment of containment measures. Individuals infected with COVID-19 exhibit disease symptoms about 2 to 14 days after infection.

The virus has been detected in respiratory secretions, which are considered as the primary means of transmission. Once viral particles enter the respiratory tract, the virus attaches to pulmonary cells via the ACE-2 receptors and are then endocytosed. SARS-CoV-2 can also be transmitted via the fecal route.

Patients positive for SARS-CoV-2 and that are symptomatic are diagnosed with COVID-19. Symptoms can vary drastically and notably include fever, dry cough, anosmia, sputum production, headaches, dyspnea, fatigue, nausea, and diarrhea. While some cases can be asymptomatic, others can lead to acute respiratory distress syndrome (ARDS) that is associated with a “cytokine storm” and even death.

Diagnosis mainly relies on real-time reverse transcription polymerase chain reaction (RT-PCR) testing of respiratory specimens. However, RT-PCR can lead to false negative results due to low viral loads or unsuitable collection, handling, and storage of swabs (oropharyngeal or nasopharyngeal), or failure during the reaction process. Imagery techniques, such as computed tomography (CT), can also be performed and show bilateral multilobular ground-class lung opacities to aid in diagnosis.

Serological testing plays a critical role in understanding and combating the viral outbreaks. Serological testing can provide robust epidemiological data that are invaluable in determining the rates of infection and thus true mortality metrics. In addition, it can aid in determining individuals that mount a robust immune response who then can be donors for therapeutics agents, such as immune (convalescent) plasma. Serological testing can also help determine the immune response of asymptomatic individuals. This is especially useful for healthcare and emergency response workers as it could help limit risk and inform staffing decisions.

Research is on-going to understand the antibody response to this virus as well as if the antibody response in individuals will confer immunity. Early studies from China show an antibody response starts to develop in the early weeks of infection (7 to 30 days post infection). It is important to understand the timing of antibody production and seroconversion in addition to understanding when and if class switching occurs. Serological class switching is the conversion of B cell antibody production from one isotype of antibody to another (or example, IgM to IgG class switching). It is important to understand the transition from the IgM isotype antibody, which are generated in the acute phase of the disease to the IgG isotype, which are present in the convalescent phase of the disease and generally confer immunity.

The SARS-CoV-2 genome codes for four main structural proteins: spike (S), envelope (E), nucleoprotein (N) (also known as the “N” protein or nucleocapsid protein) and membrane (M) proteins. The spike glycoproteins were found to bind to the ACE-2 receptors for entry into the cell. Based on research from the SARS-CoV and MERS-CoV viruses, antigen presentation stimulates the humoral and cellular immunity. These viruses exhibit a typical IgM and IgG production pattern. IgM antibodies typically disappear at the end of 12 weeks, while IgG antibodies persist which suggests that the antibodies may play protective role. Studies indicate that IgG antibodies are primarily to the S and N proteins. The spike protein has two primary subunits: subunit 1 (S1) which includes the Receptor Binding Domain (RBD) that attaches the virus to the cell membrane, binding to the human ACE2 receptor. Subunit 2 (S2) mediates the fusion of the virus and cellular membranes.

### BRIEF SUMMARY

The present disclosure relates to the development of novel immunoassays for the detection of SARS-CoV-2 antigens and/or SARS-CoV-2 specific antibodies and, optionally, one or more cytokine in patient samples. The immunoassays can be performed via a standard immunoassay format or on an automated platform. In various embodiments, the immunoassays use one or more of the following SARS-CoV-2 structural proteins (antigens): spike (S), S1, S2, envelope (E), nucleocapsid (N) and/or membrane (M) proteins or fragments thereof. As an example, a fragment of the S1 protein is the RBD portion of the S1 protein. The SARS-CoV-2 structural proteins can be immobilized on a substrate and used for the detection of antibodies in biological samples obtained from subjects. Other embodiments provide immunoassays that use one or more of the following SARS-CoV-2 structural proteins (antigens): spike (S), S1, S2, spike protein variants, S1 variants, RBD variants, S2 variants, envelope (E), nucleocapsid (N) and/or membrane (M) proteins or fragments thereof. These methods permit the quantitative, semi-quantitative, and/or qualitative detection of antibodies to these antigens as well as a means to differentiate between SARS-CoV-2 infected subjects and subjects vaccinated with vaccines based on spike proteins. Yet other embodiments provide immunoassays utilizing antibodies specific for SARS-CoV-2 structural proteins. Other embodiments provide for an immunoassay that detects SARS-CoV-2 antigen(s) and/or antibodies specific for SARS-CoV-2 structural proteins (antigens) and, optionally, one or more cytokine in a biological sample. In some embodiments, it is possible to distinguish between vaccinated individuals receiving mRNA spike protein specific vaccines or S1/S2 specific protein vaccines and individuals infected by SARS-CoV-2 on the basis of antibodies that bind to the nucleoprotein (also known as nucleocapsid or “N” protein). The disclosed immunoassays are also effective in detecting antibodies generated by SARS-CoV-2 variants, such as P1 (Brazil), B.1.1.7 (UK) and B.1.351 (South Africa). In various embodiments, the immunoassays can use labeled antibodies (e.g., anti-human IgG-PE and/or IgM-PE reporters that bind to IgG, IgA, and/or IgM antibodies in the tested biological sample) to detect and/or quantify SARS-CoV-2 specific antibodies in the biological sample (e.g., a serum sample from a subject). Other aspects of the disclosure provide antigen/substrate or antibody/substrate combinations for use in the immunoassays described herein.

### BRIEF DESCRIPTION OF THE FIGURES

FIG. 1 illustrates Format A and shows antibody detection using a single anti-human Ig. Anti-human IgG detection is illustrated in the figure, but IgA and/or IgM antibodies can be detected using anti-human IgA and anti-human IgM. When using anti-human IgA and/or anti-human IgM antibodies, a sample diluent that would adsorb human IgG can be employed.

FIG. 2 illustrates Format B and shows total antibody detection using a combination of multiple anti-human Ig conjugates for detection of human IgG, IgA and IgM.

FIG. 3 illustrates Format C and shows total antibody detection that employs the use of antigen on the solid surface but also for detection of bound antibody. Biotinylated antigen binds to available antibody sites for bound antibody IgA, IgM or IgG. Streptavidin-PE binds which binds biotin is used for detection.

FIG. 4 illustrates Format D and shows antigen detection that uses a sandwich immunoassay format. Antigen detection can also be combined with total antibody detection as shown in FIG. 3. FIG. 5 illustrates Format E and shows an immunoassay that detects total antibody plus antigen.

FIG. 6 shows simultaneous detection of multiple SARS-CoV-2 analytes and uses a variety of biotinylated conjugates (Conjugate 1) to detect the binding of antibodies and/or SARS-CoV-2 antigen the bead reagents.

FIG. 7 illustrates simultaneous (multiplex) detection of anti-SARS-CoV-2 antibodies and the cytokine IL-6.

FIG. 8 demonstrates the ability of the disclosed assay to detect the variants of interest (VOI) or variants of concern (VOC). The box plot shows minimum, maximum and median IgG antibody levels (U/mL) as measured by their reactivity to recombinant proteins immobilized on the magnetic bead surfaces. Bead signals are calibrated independently for each protein and processed into individual antibody index (AI) values. Variant L452R represents a California variant.

FIG. 9 provides an illustration of sero-reactivity of a WHO standard and reference panel to trimeric wild type, B1.1.7 (UK variant; HV69-70 del, Y144del, N501Y, A570D, D614G, P681H, T7161, S982A, D1118H), B1.351 (South African variant; L18F, D80A, D215G, LAL 242-244 del, R246I, K417N, E484K, N501Y, D614G, A701V) and P1 (Brazilian variant; L18F, T20N, P26S, D138Y, R190S, K417T, E484K, N501Y, D614G, H655Y, T1021I, V1176F) spike proteins.

### DETAILED DESCRIPTION

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

The present disclosure may refer to items, such as labels, solid supports, beads, analytes, etc. according to number or letter (e.g., Detectable label 1, bead (ii), etc.). Where this nomenclature is used, these numbers and letters are meant to distinguish the item from other items of the same type (e.g., bead (i) vs. bead (ii)), and are not meant to associate a specific property with the number or letter. Any methods, devices and materials similar or equivalent to those described herein can be used in the practice of the disclosed methods, devices, and/or methods. The following definitions are provided to facilitate understanding of certain terms used frequently herein and are not meant to limit the scope of the present disclosure.

Multiplex assays are analyses that simultaneously measure the levels of more than one analyte in a single sample. Multiplex assay methods and reagents are described, e.g., in U.S. Pat. No. 6,872,578 and WO2008148883 (each of which is hereby incorporated by reference in its entirety). In the context of this application, the analytes to be measured are antibodies specific to the peptides or proteins affixed to the solid substrates disclosed herein. In preferred embodiments, the antibodies are of the IgG and/or IgM classes of antibodies. Optionally, additional analytes to be measured are antigens specific for antibodies affixed to the solid substrates disclosed herein.

The term “solid support” or “substrate” (and grammatical equivalents of these terms) are used to denote a solid inert surface or body to which an agent, such as an antibody or a peptide or protein can be immobilized. These terms (“solid support” or “substrate” (and grammatical equivalents of these terms)) may be used interchangeably. Non-limiting examples of a solid support or substrate include plastic, polystyrenes, nitrocellulose, membranes, chips, and particles. If solid supports other than particles are used, for instance, glass, polymeric or silica chips (such as microchips), plates, slides, etc., the peptides and/or proteins (target analytes) disclosed herein can be immobilized on the surface of the support at specific locations (e.g., in specific wells of a plate (e.g., microtiter plate) or at specific locations on a chip, microchip, plate or slide). Thus, it is possible to differentiate antibodies within a sample by the location at which specific binding between antibodies in a sample and the peptides and/or proteins disclosed herein occurs on the surface of the support.

Alternatively, lateral flow immunoassays can be performed in a manner analogous to those disclosed in U.S. Pat. Nos. 5,851,776 and 6,777,190 (each of which is hereby incorporated by reference in their entireties and which relate to lateral flow chromatographic assays on a membrane or other porous or non-porous materials). As discussed above, peptides and/or proteins (target analytes) disclosed herein are immobilized at discrete locations on the membrane or other porous or non-porous material (each location being specific for a particular antigen/target analyte) and then contacted with a biological sample containing antibodies that specifically bind to the disclosed peptides and/or proteins (target analytes). The specific binding to antibodies to the peptides and/or proteins that are immobilized at discrete locations on the membrane or other porous or non-porous material is then detected using conventional methods. The term “particle” is used herein to refer to a solid or semisolid body, often with linear dimensions on the micron scale (i.e., less than about 100 microns), of any shape or surface texture. Except as noted, the term is used interchangeably with “particle,” which refers to a micron scale particle, and “bead,” which refers to particles that are spherical or near-spherical in shape, often polymeric in composition. Where used in this application, the terms “particle” and “bead” (and grammatical equivalents of these terms) can be interchanged without altering the context of the passages within this application).

The term “immobilized” as used herein denotes a molecular-based coupling that is not significantly de-coupled under the conditions imposed during the steps of the assays described herein. Such immobilization can be achieved through a covalent bond, a non-covalent bond, an ionic bond, an affinity interaction (e.g., avidin-biotin or polyhistidine-Ni⁺⁺), or any other chemical bond.

Immobilization of the various combinations of peptides and/or proteins disclosed in this application can be performed by covalent or non-covalent immobilization on a substrate. For example, non-covalent immobilization can be non-specific (e.g., non-specific binding of a combination of one or more peptides and/or proteins to a polystyrene surface). Specific or semi-specific binding to a substrate can be achieved by the peptide and/or protein having a moiety that enables covalent or non-covalent binding of the peptide and/or protein to the substrate that is coated with a ligand that binds to the moiety. For example, the moiety can be a biotin or biotinyl group or an analogue thereof bound to an amino acid group of the peptide, such as 6-aminohexanoic acid, and the ligand is then avidin, streptavidin or an analogue thereof. Alternatively, the moiety can be a His-His-His-His-His-His peptide and the substrate can be derivatized with a Nitrilotriacetic Acid derivative (NTA) charged with Ni⁺⁺ ions.

Various substrates suitable for use in the disclosed methods include, and are not limited to, magnetic beads, polystyrene beads, latex beads, beads comprising co-polymers, such as styrene-divinyl benzene; hydroxylated styrene-divinyl benzene; polystyrene; carboxylated polystyrene; carbon black; non-activated, polystyrene or polyvinyl chloride activated glass; or epoxy-activated porous magnetic glass. In other embodiments, the substrate can be the floor or wall of a microtiter well; a filter surface or membrane (e.g., a nitrocellulose membrane or a PVDF (polyvinylidene fluoride) membrane, such as an Immobilon membrane); a hollow fiber; a beaded chromatographic medium (e.g., an agarose or polyacrylamide gel); a magnetic bead; a fibrous cellulose matrix; an HPLC matrix; an FPLC matrix; or any other suitable carrier, support or surface. In one embodiment, the disclosed SARS-CoV-2 proteins are immobilized onto polystyrene beads (microspheres), wherein each protein is immobilized onto a bead with a unique detectable physical parameter, and are analyzed by a platform capable of distinguishing the detectable physical parameter. Such assays may be referred to as “multiplex immunoassays” and are discussed in detail below.

Devices for performing specific binding assays, especially immunoassays, are known and can be readily adapted for use in the present methods. Solid phase assays, in general, are easier to perform than heterogeneous assay methods which require a separation step, such as precipitation, centrifugation, filtration, chromatography, or magnetism, because separation of reagents is faster and simpler. Solid-phase assay devices include microtiter plates, flow-through assay devices, chips, microchips, lateral flow substrates dipsticks and immunocapillary or immunochromatographic immunoassay devices.

Currently, there are three main types of COVID-19 vaccines available in the United States that have received emergency use authorization (EUA) from the United States Food and Drug Administration. The Pfizer-BioNTech and Moderna vaccines are mRNA vaccines whereas the authorized Johnson & Johnson Janssen vaccine is a viral vector vaccine. All three vaccine types are targeted towards the S1/S2 spike protein of the SARS-CoV2 virus. Thus, it is possible to distinguish between infected individuals and vaccinated individual on the basis of antibodies specific to the nucleocapsid among infected individuals.

The terms “receptacle,” “vessel,” “tube,” “well,” etc. refer to a container that can hold reagents or an assay. If the receptacle is in a kit and holds reagents, it will typically be closed or sealed. If the receptacle is being used for an assay, it will typically be open or accessible during steps of the assay.

The term “biological sample” encompasses a variety of sample types obtained from an organism. The term encompasses bodily fluids, such as blood, blood components, saliva, nasal mucous, serum, plasma, cerebro-spinal fluid (C SF), urine and other liquid samples of biological origin, solid tissue biopsy, tissue cultures, or supernatant taken from cultured patient cells. In the context of the present disclosure, the biological sample is typically a bodily fluid with detectable amounts of antibodies or virus, e.g., blood or a blood component (e.g., plasma or serum) or a nasal secretion (mucous). The biological sample can be processed prior to assay, e.g., to remove cells or cellular debris. The term encompasses samples that have been manipulated after their procurement, such as by treatment with reagents, solubilization, sedimentation, or enrichment for certain components.

The term “antibody” as used herein refers to a polypeptide encoded by an immunoglobulin gene or immunoglobulin genes, or fragments thereof, which specifically bind and recognize an analyte (antigen). The recognized immunoglobulin light chains are classified as either kappa or lambda. Immunoglobulin heavy chains are classified as gamma, mu, alpha, delta, or epsilon, which in turn define the immunoglobulin classes, IgG, IgM, IgA, IgD and IgE, respectively. An example of a structural unit of immunoglobulin G (IgG antibody) is a tetramer. Each such tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains, respectively.

Antibodies exist as intact immunoglobulins or as well-characterized fragments produced by digestion of intact immunoglobulins with various peptidases. Thus, for example, pepsin digests an antibody near the disulfide linkages in the hinge region to produce F(ab′)₂, a dimer of Fab which itself is a light chain joined to VH-CH1 by a disulfide bond. The F(ab′)₂ dimer can be reduced under mild conditions to break the disulfide linkage in the hinge region, thereby converting the F(ab′)₂ dimer into two Fab′ monomers. The Fab′ monomer is essentially an Fab with part of the hinge region (see, Paul (Ed.), Fundamental Immunology, Third Edition, Raven Press, N.Y. (1993)). While various antibody fragments are defined in terms of the digestion of an intact antibody, one of skill will appreciate that such fragments may be synthesized de novo either chemically or by utilizing recombinant DNA methodology. Thus, the term “antibody,” as used herein, also includes antibody fragments either produced by the modification of whole antibodies.

Antibodies are commonly referred to according their targets. While the nomenclature varies, one of skill in the art will be familiar and understand that several names can be applied to the same antibody. For example, an antibody specific for IgM can be called “anti-IgM,” “IgM antibody,” “anti-IgM antibody,” etc.

The terms “specific for,”, “specific to”, “specifically binds,” and grammatically equivalent terms refer to a molecule (e.g., antibody or antibody fragment) that binds to its target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. For example, an antibody that specifically binds a given antibody target will typically bind the antibody target with at least a 2-fold greater affinity than a non-antibody target. Specificity can be determined using standard methods, e.g., solid-phase ELISA immunoassays (see, e.g., Harlow & Lane, Using Antibodies, A Laboratory Manual (1998) for a description of immunoassay formats and conditions that can be used to determine specific immunoreactivity).

The term “binds” with respect to an antibody target (e.g., antigen, analyte), typically indicates that an antibody binds a majority of the antibody targets in a pure population (assuming appropriate molar ratios). For example, an antibody that binds a given antibody target typically binds to at least ²/₃ of the antibody targets in a solution (e.g., at least any of 75, 80, 85, 90, 91, 92, 93, 94, 95, 96, 97, 98, 99, or 100%). One of skill will recognize that some variability will arise depending on the method and/or threshold of determining binding.

The terms “label,” “detectable label, “detectable moiety,” and like terms refer to a composition detectable by spectroscopic, photochemical, biochemical, immunochemical, chemical, or other physical means. For example, useful labels include fluorescent dyes (fluorophores), luminescent agents, electron-dense reagents, enzymes (e.g., as commonly used in an ELISA), biotin, enzymes acting on a substrate (e.g., horseradish peroxidase), digoxigenin, ³²P and other isotopes, haptens, and proteins which can be made detectable, e.g., by incorporating a radiolabel into the peptide or used to detect antibodies specifically reactive with the peptide. The term includes combinations of single labeling agents, e.g., a combination of fluorophores that provides a unique detectable signature, e.g., at a particular wavelength or combination of wavelengths. Any method known in the art for conjugating label to a desired agent may be employed, e.g., using methods described in Hermanson, Bioconjugate Techniques 1996, Academic Press, Inc., San Diego.

The term “positive,” when referring to a result or signal, indicates the presence of an analyte or item that is being detected in a sample. The term “negative,” when referring to a result or signal, indicates the absence of an analyte or item that is being detected in a sample. Positive and negative are typically determined by comparison to at least one control, e.g., a threshold level that is required for a sample to be determined positive, or a negative control (e.g., a known blank). A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters, and will understand which controls are valuable in a given situation and be able to analyze data based on comparisons to control values. Controls are also valuable for determining the significance of data. For example, if values for a given parameter are variable in controls, variation in test samples will not be considered as significant.

A “calibration control” is similar to a positive control, in that it includes a known amount of a known analyte. In the case of a multiplex assay, the calibration control can be designed to include known amounts of multiple known analytes. The amount of analyte(s) in the calibration control can be set at a minimum cut-off amount, e.g., so that a higher amount will be considered “positive” for the analyte(s), while a lower amount will be considered “negative” for the analyte(s). In some cases, multilevel calibration controls can be used, so that a range of analyte amounts can be more accurately determined. For example, an assay can include calibration controls at known low and high amounts, or known minimal, intermediate, and maximal amounts.

The term “diagnosis” refers to a relative probability that a subject has an infection, disorder or disease. Similarly, the term “prognosis” refers to a relative probability that a certain future outcome may occur in the subject. For example, in the context of the present disclosure, prognosis can refer to the likelihood that an individual has been infected by SARS-CoV-2 and has, or will develop, disease. The terms are not intended to be absolute, as will be appreciated by any one of skill in the field of medical diagnostics.

“Subject,” “patient,” “individual” and grammatical equivalents thereof are used interchangeably and refer to, except where indicated, mammals, such as humans and non-human primates, as well as rabbits, felines, canines, rats, mice, squirrels, goats, pigs, deer, and other mammalian species. The term does not necessarily indicate that the subject has been diagnosed with a particular disease, but typically refers to an individual under medical or veterinary supervision. A patient can be an individual that is seeking treatment, monitoring, adjustment or modification of an existing therapeutic regimen, etc.

The terms “structural protein” “peptide”, “antigen”, “analyte”, and “fragment” (and grammatical equivalents thereof) can be used interchangeably and refer to the disclosed SARS-CoV-2 proteins and fragments thereof that are disclosed herein. As discussed herein, fragments of the disclosed SARS-CoV-2 proteins are between 5 and (n−1) consecutive amino acids of a given SARS-CoV-2 protein or fragment, such as the RBD of the S1 protein, where n is the length of the disclosed SARS-CoV-2 protein or fragment. The length can include or exclude signal peptides that are processed. Thus, in the case of the spike protein, the length can include or exclude the signal peptide of the protein. In some embodiments, an “analyte” may be an antibody specific for a SARS-CoV-2 structural protein (antigen). In such embodiments, the use of the term in the context of detecting the antibody as the analyte will be clear. In yet other embodiments, one or more of the following chemokines and cytokines are included as an “analyte” or “analytes” that are to be detected and/or quantified: IL-1beta, IFN-γ, IFNγ-induced protein 10 (IP-10), and monocyte chemoattractant protein 1 (MCP-1), IL-4, IL-10, IL-2R, IL-6, granulocyte colony-stimulating factor (G-CSF), macrophage inflammatory protein-1A, and TNF-α. While IL-1beta, IFN-γ, IFNγ-induced protein 10 (IP-10), and monocyte chemoattractant protein 1 (MCP-1), IL-4, IL-10, IL-2R, IL-6, granulocyte colony-stimulating factor (G-CSF), macrophage inflammatory protein-1A, and TNF-a are either classified as cytokines or chemokines (in the case of IP-10), this collection of cytokines and chemokines (IL-1beta, IFN-γ, IFNγ-induced protein 10 (IP-10), and monocyte chemoattractant protein 1 (MCP-1), IL-4, IL-10, IL-2R, IL-6, granulocyte colony-stimulating factor (G-CSF), macrophage inflammatory protein-1A, and TNF-α) can be referred to herein as “cytokines” “cytokine”, or “one or more cytokine”. As discussed above, the use of the term in the context of detecting or immobilizing these chemokines and/or cytokines (“cytokines”, “cytokine”, or “one or more cytokine”) will be clear.

As discussed above, the present disclosure relates to the development of a novel multiplex immunoassay for the detection of SARS-CoV-2 antibodies and, optionally, one or more cytokine selected from IL-1beta, IFN-γ, IFNγ-induced protein 10 (IP-10), and monocyte chemoattractant protein 1 (MCP-1), IL-4, IL-10, IL-2R, IL-6, granulocyte colony-stimulating factor (G-CSF), macrophage inflammatory protein-1A, and TNF-α in patient samples suspected of infection by the virus. The disclosed immunoassays are designed to measure antibodies (e.g., IgM, IgA and/or IgG antibodies) and, optionally, levels of one or more cytokine in such patients. The immunoassay can be performed via a standard immunoassay using ELISA, lateral flow, magnetic assays with manual or using automated platforms. Particularly, the disclosed immunoassay uses one or more SARS-CoV-2 protein, or fragment thereof, selected from the SARS-CoV-2 spike (S), S1 or the RBD of the S1 protein, S2, envelope (E), nucleocapsid (N) and/or membrane (M) proteins or fragments thereof and any combination of said proteins (e.g., any combination of two, three, four, five, or six of the antigens) and, optionally, one or more cytokine selected from IL-1beta, IFN-γ, IFNγ-induced protein 10 (IP-10), monocyte chemoattractant protein 1 (MCP-1), IL-4, IL-10, IL-2R, IL-6, granulocyte colony-stimulating factor (G-CSF), macrophage inflammatory protein-1A, and TNF-α (e.g., any combination of one, two, three, four, five, six, seven, eight, nine, ten, or eleven of these analytes). As discussed above, the disclosed immunoassays can be used to distinguish between individuals infected by SARS-CoV-2 by detecting the presence of anti-nucleocapsid antibodies in samples of individuals infected by SARS-CoV-2. In vaccinated individuals, the vaccine induces antibodies to the S1/S2 spike protein of the SARS-CoV2 virus and samples from vaccinated individuals would not be expected to contain antibodies to the nucleocapsid. However, individuals in whom “break-through” infections are observed (vaccinated individuals who are later infected with SARS-CoV-2) would be expected to generate an immune response to the nucleocapsid of SARS-CoV-2.

Another embodiment provides an immunoassay in which a single SARS-CoV-2 protein or fragment thereof is immobilized on a substrate. Thus, the disclosed immunoassay comprises contacting a substrate to which one or more SARS-CoV-2 protein or fragment thereof have been immobilized under conditions effective to bind antibodies found in a biological sample to the immobilized SARS-CoV-2 specific protein or fragment thereof. Antibodies bound to the SARS-CoV-2 protein or fragment thereof that is attached to a substrate can then be detected by a species specific anti-IgM, anti-IgG, anti-IgA, anti-IgD or anti-IgE antibody that is labeled (e.g., anti-human, anti-rabbit, anti-canine, anti-rat, anti-mouse, anti-squirrel, anti-goat, anti-pig or anti-deer antibody). For example, anti-IgG-PE and/or anti-IgM-PE reporters can be used to detect and/or quantify the SARS-CoV-2 specific antibodies in biological samples obtained from individuals suspected of infection by the virus. In certain preferred embodiments, the species specific antibodies are anti-human antibodies. In certain other embodiments, the immunoassay provides a means for distinguishing individuals infected with Sars-CoV-2 from immunized individuals on the basis of detection of anti-nucleocapsid antibodies in the sample obtained from the individual.

The protein and fragments disclosed herein can be immobilized on a solid support via covalent or non-covalent bonding. In embodiments where an antibody, cytokine or a SARS-CoV-2 protein or fragment thereof is covalently immobilized on the substrate, carboxylated substrates, such as particles, plastics, polystyrenes or beads, are activated and esterified before adding the SARS-CoV-2 protein or fragment thereof. Carboxyl activation is achieved using a water soluble carbodiimide, such as 1-cyclohexyl-3-(2-morpholinoethyl) carbodiimide (CMC). Esterification is achieved using NHS, NHSS or HOBt or other suitable reagents. After carboxyl activation and esterification, the SARS-CoV-2 proteins or fragments thereof are added to the actived surface in buffers with pH between 6-10 (an example of which is sodium acetate buffer pH 5.1, phosphate buffer pH 7.0 with or without detergent (e.g., CHAPS)). After the coupling, the substrate (e.g., beads) is blocked in buffers containing protein blockers, such as BSA, mouse IgG, bovine gamma globulin (BGG) or animal serum (goat, horse, murine). The protein blocker(s) can be present in an amount ranging from 0.1-10 weight/volume percent. The blocked antigen coupled substrates can then be washed with an appropriate buffer and used in a desired immunoassay format.

As discussed above, this disclosure is directed to an immunoassay method, which includes taking a sample of body fluid or tissue (e.g., a biological sample) likely to contain antibodies; contacting (reacting) the biological sample with a SARS-CoV-2 protein or fragment thereof a combination of SARS-CoV-2 proteins or fragment thereof (and/or one or more cytokine) under conditions effective for the formation of a specific protein-antibody complex (sometimes referred to as specific binding of the protein and the antibody or “an immunocomplex” of a given protein and antibodies that specifically bind the protein or fragment thereof); and assaying the contacted (reacted) sample for the presence of an antibody-analyte immunocomplex (e.g., determining the amount of an antibody-cytokine complex and/or the immunocomplex comprising antibody and SARS-CoV-2 proteins or fragments thereof).

In various embodiments, the disclosed method relates to a diagnostic method that comprises taking a sample of body fluid or tissue (a biological sample) likely to contain antibodies (IgG, IgE, IgD, IgM, or IgA isotypes) and detecting and/or quantifying the presence of the antibodies and/or one or more cytokine within the biological sample. Generally, IgM, IgA, and/or IgG antibodies are detected (although antibodies of other isotypes may also be detected). In various embodiments, the biological sample is a serum or plasma sample derived from a venous blood sample. Other body fluids, such as saliva, gastric secretions, nasal secretions, mucus, etc., that are known to contain antibodies may also be referred to as a biological sample and used in the disclosed immunoassays.

In various embodiments, the assay may comprise immobilizing antibody(s) in the biological sample on a substrate (forming a substrate coated with antibody), adding one or more peptide and/or protein disclosed herein to the antibody coated substrate, and then detecting antibody bound to the peptide or protein. The antibody present in the biological sample can be detected by using a labeled peptide or protein or by adding a labeled antibody that specifically binds to the peptide or protein.

In particular embodiments, multiplex immunoassays can be used to detect the presence of one or more of the SARS-CoV-2 proteins or fragments thereof and/or antibodies specific for one or more of the SARS-CoV-2 proteins or fragments thereof and, optionally, one or more cytokine selected from IL-1beta, IFN-γ, IFNγ-induced protein 10 (IP-10), monocyte chemoattractant protein 1 (MCP-1), IL-4, IL-10, IL-2R, IL-6, granulocyte colony-stimulating factor (G-CSF), macrophage inflammatory protein-1A, and TNF-α (e.g., any combination of one, two, three, four, five, six, seven, eight, nine, ten, or eleven of these analytes). Thus, the presently described immunoassays involve detection of more than one analyte in a single assay, and are, thus, referred to as multiplex assays. The presently described assays include components for immobilizing single or multiple analytes on distinguishable solid supports so that each of the analytes can be identified and quantified by flow cytometry. Assay components and considerations include the solid supports and how to distinguish the different types of solid supports from one another (e.g., labels or other differentiation parameters), components to specifically immobilize the desired analyte(s) and remove other sample materials, and labels for detecting and quantifying the desired analytes.

The presently described multiplex assays involve use of a solid support, typically particles or beads. For detection by flow cytometry, particles or beads that emit high levels of autofluorescence should be avoided since this will increase background signal and potentially render them unsuitable. Particles or beads created by standard emulsion polymerization from a variety of starting monomers generally exhibit low autofluorescence, while those that have been modified to increase porosity (“macroporous” particles) exhibit high autofluorescence. Autofluorescence in such particles or beads further increases with increasing size and increasing percentage of divinylbenzene monomer. Within these limitations, the size range of the particles or beads can vary and particular size ranges are not critical. In most cases, the aggregated size range of the particles or beads lies within the range of from about 0.3 micrometer to about 100 micrometers in particle or bead diameter, e.g., within the range of from about 0.5 micrometer to about 40 micrometers.

Magnetic particles or beads are commonly used in the art, and can make separation and wash steps more convenient for the presently described assays. “Magnetic particles,” “magnetically responsive material,” “magnetic beads,” and like terms denote a material that responds to a magnetic field. Magnetically responsive materials include paramagnetic materials (e.g., iron, nickel, and cobalt, as well as metal oxides, such as Fe₃O₄, BaFe₁₂O₁₉, CoO, NiO, Mn₂O₃, Cr₂O₃, and CoMnP), ferromagnetic materials, ferrimagnetic materials, and metamagnetic materials. Rather than constituting the entire particle or bead, the magnetically responsive material typically constitutes one component of the microparticle or bead, while the remainder consists of a polymeric material which can be chemically derivatized to permit attachment of an assay reagent (e.g., antigen/analyte or antibody).

Methods of, and instrumentation for, applying and removing a magnetic field as part of an assay are known to those skilled in the art and reported in the literature. Examples of literature reports are Forrest et al., U.S. Pat. No. 4,141,687; Ithakissios, U.S. Pat. No. 4,115,534; Vlieger et al., Analytical Biochemistry 205:1-7 (1992); Dudley, Journal of Clinical Immunoassay 14:77-82 (1991); and Smart, Journal of Clinical Immunoassay 15:246-251 (1992).

The polymeric matrix that forms the microparticle or bead can be any material that is compatible with the presently described multiplex assay. The matrix should be inert to the components of the biological sample and to the assay reagents, have minimal autofluorescence, be solid and insoluble in the sample and in any other reagents or washes used in the assay, and capable of affixing an assay reagent to the microparticle. Examples of suitable polymers are polyesters, polyethers, polyolefins, polyalkylene oxides, polyamides, polyurethanes, polysaccharides, celluloses, and polyisoprenes. Crosslinking is useful in many polymers for imparting structural integrity and rigidity to the microparticle.

Functional groups for attachment of the assay reagent (e.g., antigen/analyte or antibody) can be incorporated into the polymer structure by conventional means. Examples of suitable functional groups are amine groups, ammonium groups, hydroxyl groups, carboxylic acid groups, and isocyanate groups. The assay reagent is typically covalently bound to the solid phase surface, either directly or indirectly, e.g., with a linking group. Linking groups can be used as a means of increasing the density of reactive groups on the solid phase surface and decreasing steric hindrance to increase the range and sensitivity of the assay, or as a means of adding specific types of reactive groups to the solid phase surface to broaden the range of types of assay reagents that can be affixed to the solid phase. Examples of suitable useful linking groups are polylysine, polyglycine, polyaspartic acid, polyglutamic acid and polyarginine.

Particles or beads of different types in a multiplex assay can be distinguished from one another, e.g., by size, weight, light scatter or absorbance, reflectance, shape, or label, e.g., fluorescent (dye) label. Where particle or bead size is used as a differentiation factor (distinguishing characteristic), the widths of the size subranges and the spacing between mean diameters of adjacent subranges are selected to permit differentiation of different types of particles or beads by flow cytometry, as will be apparent to those skilled in the use of and instrumentation for flow cytometry. Typically, a subrange for a given mean diameter is about ±5% CV or less of the mean diameter, where CV is the coefficient of variation and is defined as the standard deviation of the particle or bead diameter divided by the mean particle diameter times 100 percent. The mean diameters of subranges for different types of particles are generally spaced apart by at least about 6% of the mean diameter of one of the subranges, e.g., at least about 8% or 10% of the mean diameter of one of the subranges.

Light scatter can also be used to distinguish different types of particles or beads. Side angle light scatter varies with particle or beads size, granularity, absorbance and surface roughness, while forward angle light scatter is mainly affected by size and refractive index. Varying any of these qualities can result in light scatter differences that can serve as a means of distinguishing the various groups or particles or beads.

Still another example of a differentiation parameter is absorbance. When light is applied to particles or beads, the absorbance of the light by the particles or beads is indicated mostly by a change in the strength of the laterally (side-angle) scattered light while the strength of the forward-scattered light is relatively unaffected. Consequently, the difference in absorbance between various colored dyes associated with the particles or beads is determined by observing differences in the strength of the laterally scattered light.

Other physical parameters that can be used as differentiation parameters to distinguish the particles or beads of one group from those of another include excitable fluorescent dyes or colored dyes that impart different emission spectra and/or scattering characteristics to the particles or beads. Alternatively, different concentrations of one or more fluorescent dyes can be used for distinguishing or differentiating particles or beads.

When the distinguishable characteristic is a fluorescent dye or color, it can be coated on the surface of the particle or bead, embedded in the particle or bead, or bound to the molecules of the particle or bead material. Thus, fluorescent particles or beads can be manufactured by combining the polymer material with the fluorescent dye, or by impregnating the particle or bead with the dye. Particles or beads with dyes already incorporated and thereby suitable for use in the present invention are commercially available, from suppliers, such as Spherotech, Inc. (Libertyville, Ill., USA) and Molecular Probes, Inc. (Eugene, Oreg., USA).

Labels can be any substance or component that directly or indirectly emits or generates a detectable signal. In some embodiments, the labels are fluorophores, many of which are reported in the literature and thus known to those skilled in the art, and many of which are readily commercially available. Literature sources for fluorophores include Cardullo et al., Proc. Natl. Acad. Sci. USA 85: 8790-8794 (1988); Dexter, J. of Chemical Physics 21: 836-850 (1953); Hochstrasser et al., Biophysical Chemistry 45: 133-141 (1992); Selvin, Methods in Enzymology 246: 300-334 (1995); Steinberg, Ann. Rev. Biochem., 40: 83-114 (1971); Stryer, Ann. Rev. Biochem. 47: 819-846 (1978); Wang et al., Tetrahedron Letters 31: 6493-6496 (1990); and Wang et al., Anal. Chem. 67: 1197-1203 (1995). The following are non-limiting examples of fluorophores that can be used as labels:

4-acetamido-4′-isothiocyanatostilbene-2,2′disulfonic acid; acridine; acridine isothiocyanate; 5-(2′-aminoethyl)aminonaphthalene-1-sulfonic acid (EDANS); 4-amino-N-[3-vinylsulfonyl)phenyl]naphthalimide-3,5di sulfonate; N-(4-anilino-1-naphthyl)maleimide; anthranilamide; BODIPY; Brilliant Yellow; coumarin; 7-amino-4-methylcoumarin (AMC, Coumarin 120); 7-amino-4-trifluoromethylcoumarin (Coumaran 151); cyanine dyes;

cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-(4′-dimethylaminophenylazo)benzoic acid (DABCYL); 4-dimethylaminophenylazophenyl-4′-isothiocyanate (DABITC); eosin; eosin isothiocyanate; erythrosin B; erythrosin isothiocyanate; ethidium; 5-carboxyfluorescein (FAM); 5 -(4,6-dichlorotriazin-2-yl)aminofluorescein (DTAF); 2′,7′-dimethoxy-4′5′-dichloro-6-carboxyfluorescein (JOE); fluorescein; fluorescein isothiocyanate; fluorescamine; IR144; IR1446; Malachite Green isothiocyanate; 4-methylumbelliferone; ortho cresolphthalein; nitrotyrosine; pararosaniline; Phenol Red; phycoerythrin ((PE) including but not limited to B and R types); o-phthaldialdehyde; pyrene; pyrene butyrate; succinimidyl 1-pyrene butyrate; quantum dots; Reactive Red 4 (Cibacron™ Brilliant Red 3B-A); 6-carboxy-X-rhodamine (ROX); 6-carboxyrhodamine (R6G); lissamine rhodamine B sulfonyl chloride rhodamine; rhodamine B; rhodamine 123; rhodamine X isothiocyanate; sulforhodamine B; sulforhodamine 101; sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; and lanthanide chelate derivatives.

Particular fluorophores for use in the disclosed immunoassays include fluorescein, fluorescein isothiocyanate, phycoerythrin (PE), rhodamine B, and Texas Red (sulfonyl chloride derivative of sulforhodamine 101). Any of the fluorophores in the list preceding this paragraph can be used in the presently described assays, either to label the particle or bead, or to label a binding agent (e.g., an antibody or streptavidin). Fluorochromes can be attached by conventional covalent bonding, using appropriate functional groups on the fluorophores and on the particle or bead or binding agent (e.g., an antibody or streptavidin). The recognition of such groups and the reactions to form the linkages will be readily apparent to those skilled in the art. Other labels that can be used in place of the fluorophores are radioactive labels and enzyme labels. These are likewise known in the art. Flow cytometry methods and instrumentation are known in the art. Descriptions of instrumentation and methods can be found, e.g., in Introduction to Flow Cytometry: A Learning Guide (2000) Becton, Dickinson, and Company; McHugh, “Flow Microsphere Immunoassay for the Quantitative and Simultaneous Detection of Multiple Soluble Analytes,” Methods in Cell Biology 42, Part B (Academic Press, 1994).

This disclosure also pertains to kits and compositions for the detection of SARS-CoV-2 infection in a subject. The multiplex assay disclosed herein provides for the detection and/or quantification of immunoglobulin G (IgG) antibodies, IgA, and/or IgM antibodies specific for SARS-CoV-2 proteins or fragments thereof. The multiplex assay disclosed herein can detect and/or quantify the amount of specific IgG, IgA and/or IgM antibodies and, optionally, one or more cytokine selected from IL-1beta, IFN-γ, IFNγ-induced protein 10 (IP-10), monocyte chemoattractant protein 1 (MCP-1), IL-4, IL-10, IL-2R, IL-6, granulocyte colony-stimulating factor (G-CSF), macrophage inflammatory protein-1A, and TNF-α (e.g., any combination of one, two, three, four, five, six, seven, eight, nine, ten, or eleven of these analytes) present in a biological sample. The multiplex assays disclosed herein can also detect the presence of total amount of SARS-CoV-2 specific antibody and, optionally, one or more cytokine selected from IL-1beta, IFN-γ, IFNγ-induced protein 10 (IP-10), monocyte chemoattractant protein 1 (MCP-1), IL-4, IL-10, IL-2R, IL-6, granulocyte colony-stimulating factor (G-CSF), macrophage inflammatory protein-1A, and TNF-α (e.g., any combination of one, two, three, four, five, six, seven, eight, nine, ten, or eleven of these analytes) present in a biological sample.

Various of the presently described assays offer detection in at least two dimensions, e.g., the identity of the immobilizing bead (e.g., beads bearing a single SARS-CoV-2 specific peptide or protein or any combination of the SARS-CoV-2 specific peptides and/or proteins disclosed herein, and the presence and amount of antibody bound to the SARS-CoV-2 specific peptide and/or proteins immobilized on the beads and, optionally, one or more cytokine selected from IL-1beta, IFN-γ, IFNγ-induced protein 10 (IP-10), monocyte chemoattractant protein 1 (MCP-1), IL-4, IL-10, IL-2R, IL-6, granulocyte colony-stimulating factor (G-CSF), macrophage inflammatory protein-1A, and TNF-α (e.g., any combination of one, two, three, four, five, six, seven, eight, nine, ten, or eleven of these analytes). This multidimensional aspect allows for a multiplex format, so that more than one analyte can be detected in a single assay.

Thus, one aspect provides for combinations of substrate populations. These substrate combinations are made up of two or more (e.g., two, three, four, five, six, seven or more) distinct and unique detectable physical parameters (e.g., dye signatures), each distinct and unique detectable physical parameter being associated with a single substrate population.

In some embodiments, the presence and amount of each of the antibody isotypes and, optionally, one or more cytokine are measured in the same single receptacle or vessel (tube, well, cuvette, etc.) in the presence of beads. As discussed above, each bead population carries a specific detectable physical parameter (e.g., dye signature), and a SARS-CoV-2 specific protein or fragment thereof. Thus, the beads can carry a single peptide or protein selected from spike (S), S1, S2, envelope (E), nucleocapsid (N) and/or membrane (M) proteins and/or fragments thereof, such as the RBD of the S1 protein or a fragment thereof or monoclonal or polyclonal antibodies specific for one or more selected cytokine.

In certain aspects, the SARS-CoV-2 immunoassay will employ the use of the Spike and nucleocapsid proteins or fragments of these two proteins and, optionally, antibodies specific to one of more cytokine. The proteins or fragments thereof can include, but is not limited to, the SARS-CoV-2 Spike Glycoprotein S1, the RBD portion of the S1 protein, Spike Glycoprotein S2, and the nucleocapsid protein and can be immobilized on a solid substrate (for example, a bead). In addition, the assay may employ the use of an antibody coated beads for the detection of viral antigen and, optionally, antibodies specific to one or more cytokine. The antibodies detect the Spike protein or fragments thereof (e.g., S1, the RBD of the S1 protein and/or S2) and/or the nucleocapsid protein or fragments thereof and, optionally, one or more selected cytokine. Certain embodiments of the immunoassay will employ the use of a multiplex flow immunoassay, a methodology that greatly resembles traditional EIA, but permits simultaneous detection and identification of antibodies to multiple proteins in a single tube.

### Coupling/Ommobilization Antibodies and Recombinant Protein Antigens

The antibodies (e.g., cytokine specific antibodies and/or antibodies specific to a SARS-CoV2 protein antigen or fragment thereof) and protein antigens (e.g., SARS-CoV-2 S, S1, the RBD portion of the S1 protein or a fragment thereof, S2, nucleocapsid or combinations thereof) can be immobilized on a solid support (e.g., microparticles, beads, or surface, such as a chip, microtiter plate, membrane, or glass). In some immobilization protocols, carboxylated beads were activated and esterified before adding antibodies and/or SARS-CoV-2 specific protein antigen(s). Carboxyl activation was achieved using a water soluble carbodiimide, such as 1-cyclohexyl-3-(2-moipholinoethyl) carbodiimide (CMC). Esterification was achieved using NHS, NHSS or HOBt. After the carboxyl activation and esterification, the antibodies and SARS-CoV-2 protein antigen(s) were added to the activated surface in buffers with pH between 6-10. For example sodium acetate buffer pH 5.1, phosphate buffer pH 7.0 with or without detergent (e.g., CHAPS, ionic and zwitterionic detergents). After coupling/immobilization, the solid support was blocked in buffers containing protein blocker (such as BSA, mouse IgG, bovine gamma globulin (BGG), animal serum (goat, horse, murine)). The protein blocker(s) were present in amounts ranging from 0.1-10 weight/volume percent.

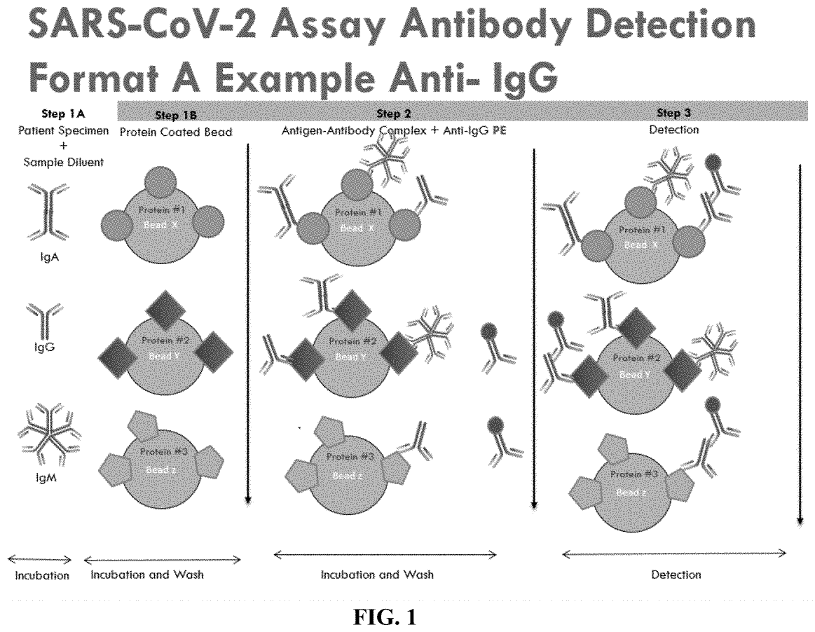

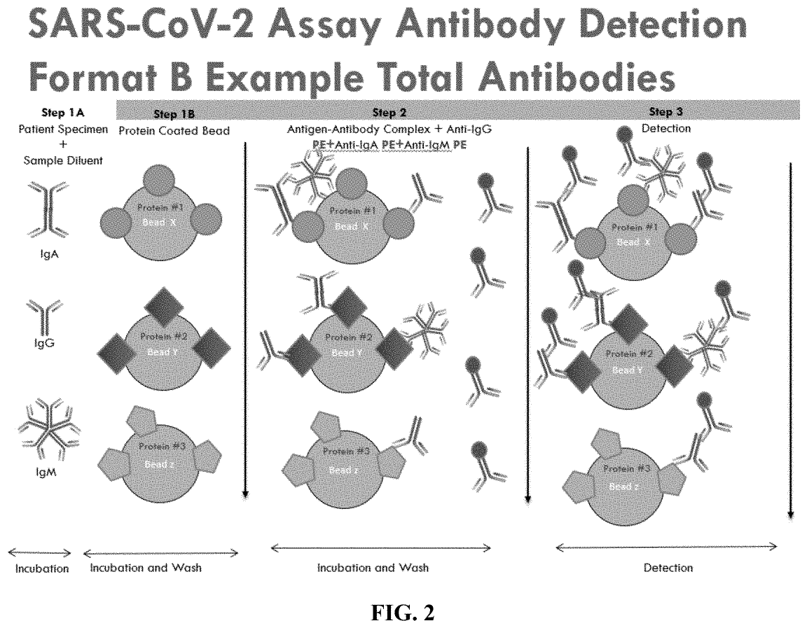

A first aspect and second provides a pair of immunoassays identified as FORMAT A (the first aspect) and FORMAT B (the second aspect). These immunoassay formats provide for the detection of SARS-CoV-2 antibodies individually or the detection of total IgG, IgM and/or IgA and, optionally, one or more cytokine. In these immunoassays, a solid support (for example, fluorescently labeled beads (also referred to as dyed beads) that can be distinguished from one another via a different fluorescent dye/signature) can be coated with protein antigens (for example, spike (S), S1, the RBD portion of the S1 protein, S2, envelope (E), nucleocapsid (N) and/or membrane (M) proteins or fragments thereof) and, optionally, one of more cytokine either individually or in different combinations as described above, the coated solid support(s) are then combined with a patient sample, e.g., a biological sample from an individual suspected of having been exposed to SARS-CoV-2 virus along with a sample diluent. In the case of IgA or IgM detection, the assays employ a sample diluent that would help block the binding of human IgG to the protein antigens thereby increasing the detection of IgA or IgM or the sample can be pretreated with a reagent that removes IgG from the sample (e.g., magnetic beads coated with anti-IgG antibodies or with a buffer comprising anti-IgG antibodies (e.g., a sample diluent containing buffer components, such as Triethanolamine 50 mM, Sodium Chloride 150 mM, Magnesium Chloride 50 mM, Proteins, such as 2% BSA, 3 mg/ml Goat Anti-Human IgG, and Preservatives, such as Sodium Azide)). After incubation unbound sample can be washed away and labeled anti-Ig (Anti IgG, IgM, or IgA) antibodies can be added to allow for detection of antibodies from the sample bound to the solid support. For example, a mixture of anti-human IgG and anti-human IgM, anti-human IgA antibodies, or an antibody that binds IgA, IgG and IgM, can be used (see FIG. 1 and FIG. 2). Where one or more cytokine is being detected or quantified in combination with of SARS-CoV-2 antibodies, labeled antibodies specific to the selected cytokine(s) can be used to detect the presence or quantify the cytokine(s). The reaction can be incubated and washed prior to detection using a BioPlex 2200, Bioplex 200 or Luminex platforms, such as LX-200, Magpix, Flexmap 360, etc. The identity of each assay is determined by the fluorescence signature of the dyed beads, and the amount of antibody captured by the antigen or cytokine is determined by the fluorescence intensity of the attached labeled anti-Ig. The sample fluorescence intensity is compared to a set of standards or calibrators to generate a qualitative, semi-quantitative or quantitative result. Results for each coated protein may be reported individually, as a group or some type of predefined algorithm or any combination thereof.

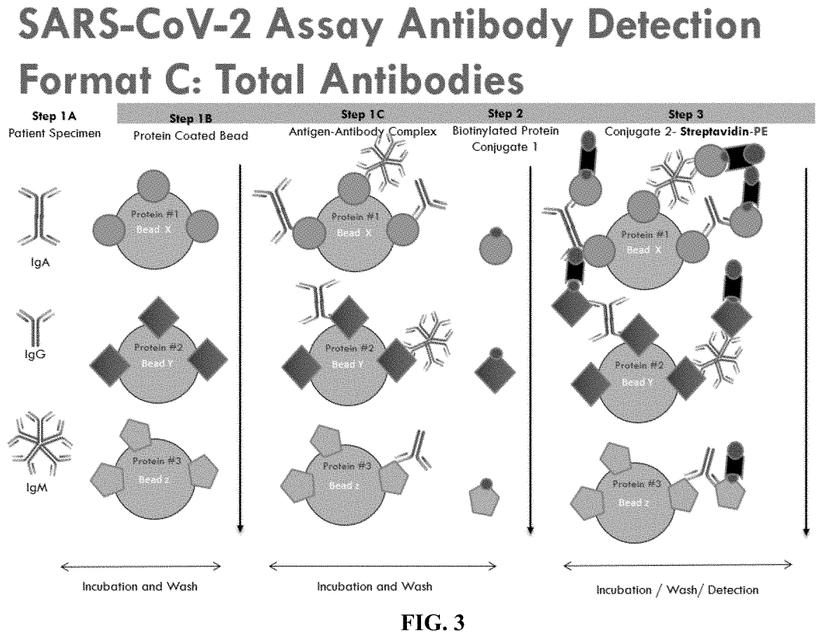

A third aspect provides an immunoassay identified as FORMAT C for the detection of SARS-CoV-2 total antibodies and, optionally, one or more cytokine. In this immunoassay, a solid support (for example, fluorescently labeled beads (also referred to as dyed beads) that can be distinguished from one another via a different fluorescent dye/signature) can be coated with protein antigens (for example, spike (S), S1, the RBD portion of the S1 protein, S2, envelope (E), nucleocapsid (N) and/or membrane (M) proteins or fragments thereof) and, optionally, one or more cytokine either individually or in different combinations as described above, the coated solid support(s) would be combined with patient sample, e.g., a biological sample from an individual suspected of having been exposed to SARS-CoV-2 virus. After incubation unbound sample was washed away and biotinylated protein specific for each immunoglobulin class (for example, biotinylated antibody specific for each class of antibody (IgM, IgA, and IgG) or biotinylated Protein A or G which binds to IgG antibody) can be added to the washed beads and incubated. Where one or more cytokine is being detected or quantified in combination with SARS-CoV-2 total antibody, biotinylated antibodies specific to the selected cytokine(s) can be used to detect the presence or quantify the cytokine(s). Unbound biotinylated protein is then washed away and labeled streptavidin-PE (Phycoerythrin) is added to allow for detection of total bound antibodies (FIG. 3). The reaction is incubated and then washed prior to detection using a BioPlex 2200, Bioplex 200 or Luminex platforms, such as LX-200, Magpix, Flexmap 360, etc. The identity of each assay (protein or cytokine coated bead) is determined by the fluorescence signature of the dyed beads, and the amount of total antibody and selected cytokine captured by the coated protein is determined by the fluorescence intensity of the labeled Streptavidin bound to biotinylated antibody. The sample fluorescence intensity is compared to the fluorescence intensity of a set of standards or calibrators to generate a qualitative, semi-quantitative or quantitative result. Results for each coated protein may be reported individually, as a group or some type of predefined algorithm or any combination thereof.

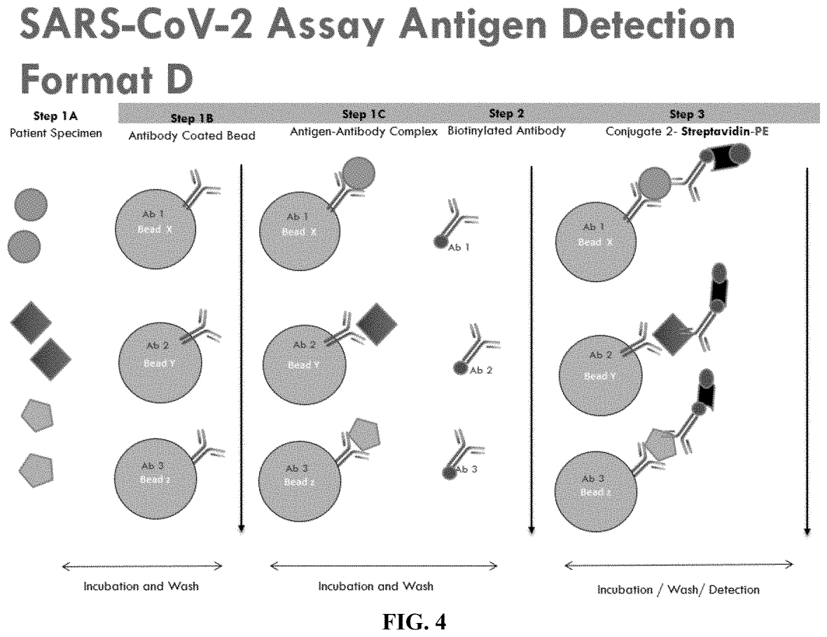

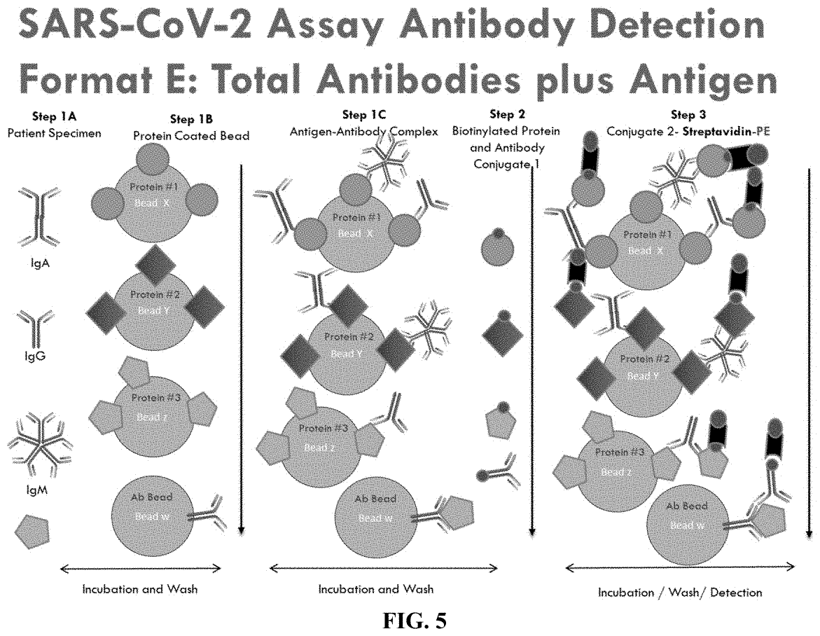

A fourth aspect provides an immunoassay identified as FORMAT D for the detection of SARS-CoV-2 antigen and, optionally, one or more cytokine. In this aspect , a solid support is coated with monoclonal and/or polyclonal antibodies that bind selected antigens (for example, spike (S), S1, the RBD portion of the S1 protein, S2, envelope (E), nucleocapsid (N) and/or membrane (M) proteins or fragments thereof) and, optionally, monoclonal and/or polyclonal antibodies specific for one or more cytokine. The antibody coated solid support(s), for example, fluorescently labeled beads (also referred to as dyed beads) that can be distinguished from one another via a different fluorescent dye/signature) can be combined with patient sample, e.g., a biological sample from an individual suspected of having been exposed to SARS-CoV-2 virus. After incubation unbound sample is washed away and biotinylated antibody for each selected antigen and, optionally one or more cytokine is added allowed to incubate. The biotinylated antibody binds to the antibody bound protein and/or cytokine (when being assayed). Unbound biotinylated antibody is then washed away and labeled streptavidin-PE (Phycoerythrin) is added to allow for detection of total bound proteins (FIG. 4). The reaction is incubated and then washed prior to detection using a BioPlex 2200, Bioplex 200 or Luminex platforms, such as LX-200, Magpix, Flexmap 360, etc. The identity of each assay (antibody coated bead) is determined by the fluorescence signature of the dyed beads, and the amount of total antigen captured by the coated antibody is determined by the fluorescence intensity of the bound labeled Streptavidin. The sample fluorescence intensity is compared to the fluorescence intensity of a set of standards or calibrators to generate a qualitative, semi-quantitative or quantitative result. Results for each coated protein may be reported individually, as a group or some type of predefined algorithm or any combination thereof. In certain embodiments, the FORMAT D immunoassay may be combined with the antibody assay of FORMAT C by combining subpopulations of beads from FORMAT C and FORMAT D and using appropriate detection reagents as set forth for each respective format.

Another format suitable for the detection of SARS-CoV-2 antigens is illustrated in

FIG. 5. In this format, one or more subpopulation of beads comprising a SARS-CoV-2 structural protein (for example, three subpopulations of beads to which RBD, N protein and S2 antigen have been immobilized) are mixed with one or more subpopulation of beads to which antibodies specific to SARS-CoV-2 antigens are immobilized (for example, a subpopulation of beads to which antibodies specific to RBD are immobilized). Each subpopulation of beads are fluorescently labeled so that they can be distinguished from one another via a different fluorescent dye/signature. The mixture of beads can be combined with patient sample, e.g., a biological sample from an individual suspected of having been exposed to SARS-CoV-2 virus. After incubation unbound sample is washed away and biotinylated antibody for each selected SARS-CoV-2 antigen and biotinylated SARS-CoV-2 antigen corresponding to the SARS-CoV-2 antigen immobilized on each bead subpopulation is added allowed to incubate. The biotinylated antibody binds to SARS-CoV-2 antigen bound to a bead to which SARS-CoV-2 specific antibody is immobilized and biotinylated antigen binds to antibodies bound to antigen immobilized on a selected bead subpopulation. Unbound biotinylated antibody and unbound biotinylated SARS-CoV-2 antigen is then washed away and labeled streptavidin-PE (Phycoerythrin) is added to allow for detection of total bound antibody and selected antigen present in the sample (FIG. 5). The reaction is incubated and then washed prior to detection using a BioPlex 2200, Bioplex 200 or Luminex platforms, such as LX-200, Magpix, Flexmap 360, etc. The identity of each assay (antibody or antigen coated bead) is determined by the fluorescence signature of the dyed beads, and the amount of total antigen captured by the coated antibody is determined by the fluorescence intensity of the bound labeled Streptavidin and antigen in the sample is, likewise, detected by fluorescence. The sample fluorescence intensity is compared to the fluorescence intensity of a set of standards or calibrators to generate a qualitative, semi-quantitative or quantitative result. Results for each coated protein may be reported individually, as a group or some type of predefined algorithm or any combination thereof.

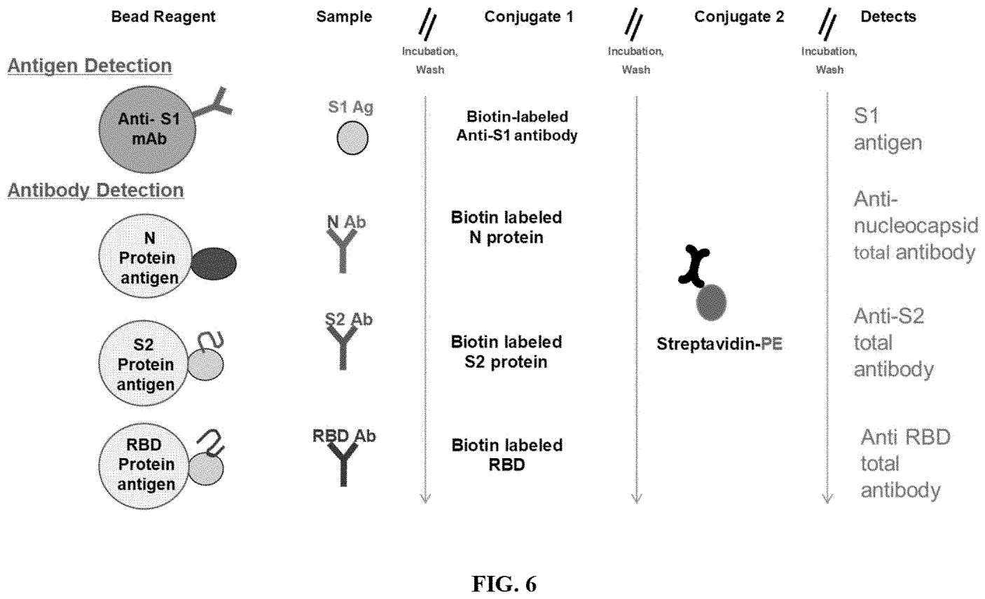

Yet another format suitable for the detection of SARS-CoV-2 antigens is illustrated in FIG. 6. In this format, one or more subpopulation of beads comprising a SARS-CoV-2 antigen (for example, three subpopulations of beads to which RBD, N protein and S2 antigen have been immobilized) are mixed with one or more subpopulation of beads to which antibodies specific to SARS-CoV-2 antigens are immobilized (for example, a subpopulation of beads to which antibodies specific to RBD are immobilized). Each subpopulation of beads are fluorescently labeled so that they can be distinguished from one another via a different fluorescent dye/signature. The mixture of beads can be combined with patient sample, e.g., a biological sample from an individual suspected of having been exposed to SARS-CoV-2 virus. After incubation unbound sample is washed away and biotinylated antibody for each selected SARS-CoV-2 antigen and biotinylated SARS-CoV-2 antigen corresponding to the SARS-CoV-2 antigen immobilized on each bead subpopulation is added allowed to incubate. The biotinylated antibody binds to SARS-CoV-2 antigen bound to a bead to which SARS-CoV-2 specific antibody is immobilized and biotinylated antigen binds to antibodies bound to antigen immobilized on a selected bead subpopulation. Unbound biotinylated antibody and unbound biotinylated SARS-CoV-2 antigen is then washed away and labeled streptavidin-PE (Phycoerythrin) is added to allow for detection of total bound antibody and identify selected antigen present in the sample (FIG. 6). The reaction is incubated and then washed prior to detection using a BioPlex 2200, Bioplex 200 or Luminex platforms, such as LX-200, Magpix, Flexmap 360, etc. The identity of each assay (antibody or antigen coated bead) is determined by the fluorescence signature of the dyed beads, and the amount of total antigen captured by the coated antibody is determined by the fluorescence intensity of the bound labeled Streptavidin and antigen in the sample is, likewise, detected by fluorescence. The sample fluorescence intensity is compared to the fluorescence intensity of a set of standards or calibrators to generate a qualitative, semi-quantitative or quantitative result. Results for each coated protein may be reported individually, as a group or some type of predefined algorithm or any combination thereof.

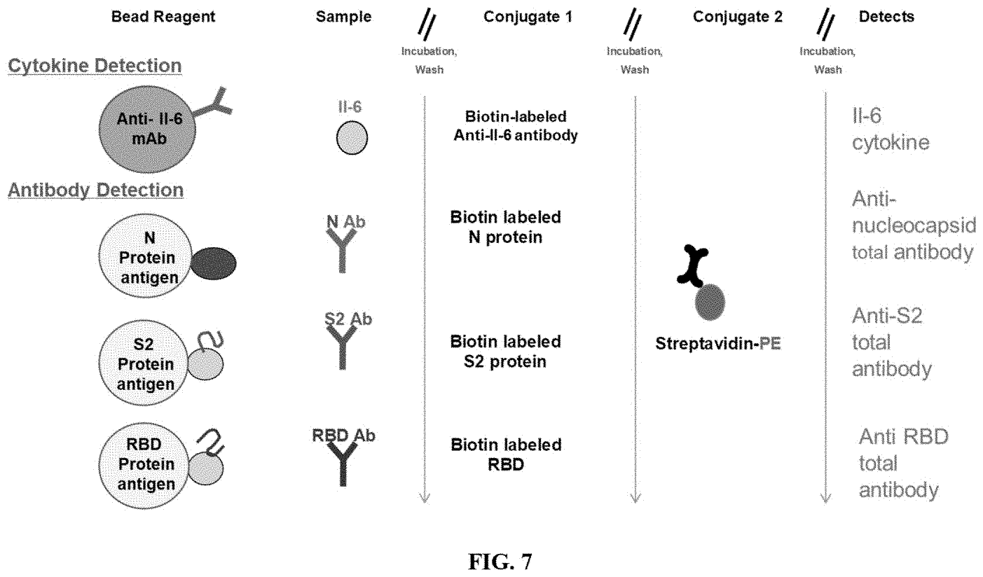

Yet another format suitable for the detection of SARS-CoV-2 antigens is illustrated in FIG. 7. In this format, one or more subpopulation of beads comprising a SARS-CoV-2 antigen (for example, three subpopulations of beads to which RBD, N protein and S2 antigen have been immobilized) are mixed with one or more subpopulations of beads to which antibodies specific to one or more cytokine selected from IL-1beta, IFN-γ, IFNγ-induced protein 10 (IP-10), monocyte chemoattractant protein 1 (MCP-1), IL-4, IL-10, IL-2R, IL-6, granulocyte colony-stimulating factor (G-CSF), macrophage inflammatory protein-1A, and TNF-α are immobilized (for example, a subpopulation of beads to which antibodies specific to IL-6 are immobilized). Each subpopulation of beads are fluorescently labeled so that they can be distinguished from one another via a different fluorescent dye/signature. The mixture of beads can be combined with patient sample, e.g., a biological sample from an individual suspected of having been exposed to SARS-CoV-2 virus. After incubation unbound sample is washed away and biotinylated antibody for each selected cytokine or chemokine and biotinylated SARS-CoV-2 antigen corresponding to the SARS-CoV-2 antigen immobilized on each bead subpopulation is added and allowed to incubate. The biotinylated antibody binds to cytokine or chemokine bound to a bead to which a cytokine or chemokine specific antibody is immobilized and biotinylated antigen binds to antibodies bound to antigen immobilized on a selected bead subpopulation. Unbound biotinylated antibody and unbound biotinylated SARS-CoV-2 antigen is then washed away and labeled streptavidin-PE (Phycoerythrin) is added to allow for detection of total bound antibody and identify selected antigen present in the sample (FIG. 7). The reaction is incubated and then washed prior to detection using a BioPlex 2200, Bioplex 200 or Luminex platforms, such as LX-200, Magpix, Flexmap 360, etc. The identity of each assay (antibody or antigen coated bead) is determined by the fluorescence signature of the dyed beads, and the amount of total antigen captured by the coated antibody is determined by the fluorescence intensity of the bound labeled Streptavidin and antigen in the sample is, likewise, detected by fluorescence. The sample fluorescence intensity is compared to the fluorescence intensity of a set of standards or calibrators to generate a qualitative, semi-quantitative or quantitative result. Results for each coated protein may be reported individually, as a group or some type of pre-defined algorithm or any combination thereof.

Another aspect provides an immunoassay for the detection of SARS-CoV-2 antigen and one or more cytokine selected from IL-1beta, IFN-γ, IFNγ-induced protein 10 (IP-10), monocyte chemoattractant protein 1 (MCP-1), IL-4, IL-10, IL-2R, IL-6, granulocyte colony-stimulating factor (G-CSF), macrophage inflammatory protein-1A, and TNF-α. In this aspect, one or more subpopulations of solid supports (beads) are coated with monoclonal and/or polyclonal antibodies that bind selected antigens (for example, spike (S), S1, the RBD portion of the S1 protein, S2, envelope (E), nucleocapsid (N) and/or membrane (M) proteins or fragments thereof) and the cytokines that are selected for detection/quantification. In this format, one or more subpopulation of beads comprising a SARS-CoV-2 antigen (for example, three subpopulations of beads to which antibodies specific to RBD, N protein and S2 antigen have been immobilized) are mixed with one or more subpopulations of beads to which antibodies specific to one or more cytokine selected from IL-1beta, IFN-γ, IFNγ-induced protein 10 (IP-10), monocyte chemoattractant protein 1 (MCP-1), IL-4, IL-10, IL-2R, IL-6, granulocyte colony-stimulating factor (G-CSF), macrophage inflammatory protein-1A, and TNF-α are immobilized (for example, a subpopulation of beads to which antibodies specific to IL-6 are immobilized). Each subpopulation of beads are fluorescently labeled so that they can be distinguished from one another via a different fluorescent dye/signature. The mixture of beads can be combined with patient sample, e.g., a biological sample from an individual suspected of having been exposed to SARS-CoV-2 virus. After incubation unbound sample is washed away and biotinylated antibody for each selected cytokine or chemokine and biotinylated antibody specific to the selected SARS-CoV-2 antigens present on each respective bead subpopulation is added and allowed to incubate. The biotinylated antibody binds to cytokine bound to a bead to which a cytokine specific antibody is immobilized and biotinylated antibodies specific to the selected SARS-CoV2 antigens bind to SARS-CoV2 antigen bound to beads on which antibodies specific to SARS-CoV2 antigens were immobilized. Unbound biotinylated antibody and unbound biotinylated SARS-CoV-2 antigen is then washed away and labeled streptavidin-PE (Phycoerythrin) is added to allow for detection and identification of cytokines and selected SARS-CoV2 antigen present in the sample. The reaction is incubated and then washed prior to detection using a BioPlex 2200, Bioplex 200 or Luminex platforms, such as LX-200, Magpix, Flexmap 360, etc. The identity of each assay (cytokine or SARS-CoV-2 antigen) is determined by the fluorescence signature of the dyed beads, and the amount of total antigen and cytokine captured is determined by the fluorescence intensity of the bound labeled Streptavidin. The sample fluorescence intensity is compared to the fluorescence intensity of a set of standards or calibrators to generate a qualitative, semi-quantitative or quantitative result. Results for each coated protein may be reported individually, as a group or some type of pre-defined algorithm or any combination thereof.

As discussed above, the each of the disclosed immunoassays can utilize SARS-CoV-2 spike (S), S1, S2, envelope (E), nucleocapsid (N) and/or membrane (M) protein or fragments thereof (for example, the RBD of the S1 protein) as an analyte. In the context, a fragment of any particular SARS-CoV-2 protein can comprise about 5 to about 50, about 10 to about 40, about 15 to about 30, about 20, about 10 or about 5 amino acids. In other embodiments, the fragments of a SARS-CoV-2 protein range in length from 5 amino acids to (n−1) consecutive amino acids of the protein, where n is the total length of a particular SARS-CoV-2 protein. Thus, for the nucleocapsid, a fragment can range between 5 and 418 consecutive amino acids of the nucleocapsid. For the spike protein, the fragment length is between 5 and 1272 consecutive amino acids in length. In one embodiment, a fragment of the spike protein spans amino acids 13-1213 of the spike protein sequence. In another embodiment, the fragment is the RBD of the S1 protein or a fragment of the RBD. For the S1 protein (amino acids 13-685 of the disclosed spike protein length), a fragment is between 5 and 672 consecutive amino acids of the S1 sequence. For the S2 protein (amino acids 686-1273 of the disclosed spike protein), the fragment length is between 5 and 588 consecutive amino acids in length. For the envelope protein, a fragment can range between 5 and 94 consecutive amino acids of the envelope protein. For the membrane protein, a fragment can range between 5 and 221 consecutive amino acids of the envelope protein. As would be apparent to those skilled in the art, fragments of different lengths of a particular protein can be coupled/immobilized to a particular solid support (e.g., solid support 1 can comprise protein fragments that are the same length, that vary in length or are a combination of varying lengths).

SARS-CoV-2 Nucleoprotein (419 AA MW 45626; also referred to
as “N” protein or nucleocapsid protein)
(SEQ ID NO: 1)
MSDNGPQNQRNAPRITFGGPSDSTGSNQNGERSGARSKQRRPQGLPNNTASWFTAL
TQHGKEDLKFPRGQGVPINTNSSPDDQIGYYRRATRRIRGGDGKMKDLSPRWYFYY
LGTGPEAGLPYGANKDGIIWVATEGALNTPKDHIGTRNPANNAAIVLQLPQGTTLPK
GFYAEGSRGGSQASSRSSSRSRNSSRNSTPGSSRGTSPARMAGNGGDAALALLLLDR
LNQLESKMSGKGQQQQGQTVTKKSAAEASKKPRQKRTATKAYNVTQAFGRRGPEQ
TQGNFGDQELIRQGTDYKHWPQIAQFAPSASAFFGMSRIGMEVTPSGTWLTYTGAIK
LDDKDPNFKDQVILLNKHIDAYKTFPPTEPKKDKKKKADETQALPQRQKKQQTVTL
LPAADLDDFSKQLQQSMSSADSTQA
SARS-CoV-2 Spike Protein (1273 AA MW 141178)
SARS-CoV-2 S1 Protein (AA 13-685)
SARS-CoV-2 S2 Protein (AA 686-1273)
(SEQ ID NO: 2)
MFVFLVLLPLVSS/QCVNLTTRTQLPPAYTNSFTRGVYYPDKVFRSSVLHSTQDLFLP
FFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRRAR/SVAS
QSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLI
ANQFNSAIGKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFPREGVF
VSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIK
WPWYIWLGFIAGLIAIVMVTIMLCCMTSCCSCLKGCCSCGSCCKFDEDDSEPVLKGV
KLHYT
SARS-CoV-2 Envelope (AA1-75)
(SEQ ID NO: 3)
MYSFVSEETGTLIVNSVLLFLAFVVFLLVTLAILTALRLCAYCCNIVNVSLVKPSFYV
YSRVKNLNSSRVPDLLV
SARS-CoV-2 Membrane (AA1-222)
(SEQ ID NO: 4)
MADSNGTITVEELKKLLEQWNLVIGFLFLTWICLLQFAYANRNRFLYIIKLIFLWLLW
PVTLACFVLAAVYRINWITGGIAIAMACLVGLMWLSYFIASFRLFARTRSMWSFNPE
TNILLNVPLHGTILTRPLLESELVIGAVILRGHLRIAGHHLGRCDIKDLPKEITVATSRT
LSYYKLGASQRVAGDSGFAAYSRYRIGNYKLNTDHSSSSDNIALLVQ
>QJF75467.1 surface glycoprotein [Severe acute respiratory
syndrome coronavirus 2]
SARS-CoV-2 Spike Protein (1273 AA MW 141178)
SARS-CoV-2 S1 Protein (AA 13-685)
SARS-CoV-2 S2 Protein (AA 686-1273)
(SEQ ID NO: 5)
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVXNATRF
ASVYAWXRKRISNCVADYSXLYNSASFSTFKCYGVSPTKLNDLCFTNVYADSFVIRG
DEVRQIAPGQTGKIADYNYKLPDDFTGCVIWNSNNLDSKVGGNYNYLYRLFRXSN
LKPFERDISTEIYQAGSTPCNGXEGFNCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRXAXSVAS
QSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLI
ANQFNSAIGKIQDSLSSTASALGKLQDVVNQNAQALNTLVKQLSSNFGAISSVLNDIL
SRLDXXEAEVQIDRLITGRLQSLQTYVTQQLIRAAEIRASANLAATKMSECVLGQSKR
VDFCGKGYULMSFPQSAPHGVVFLHVTYVPAQEKNFTTAPAICHDGKAHFPREGVF
VSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIK
WPWYIWLGFIAGLIAIVMVTIMLCCMTSCCSCLKGCCSCGSCCKFDEDDSEPVLKGV
KLHYT

Alternative methods provided in this disclosure also utilize variant SARS-CoV-2 spike proteins or fragments thereof, optionally in combination with wild-type spike (S), S1, RBD, S2, envelope (E), nucleocapsid (N) and/or membrane (M) proteins or fragments thereof for the detection of antibodies in a biological sample obtained from a subject. Where the methods seek to distinguish between vaccinated and infected individuals, the immunoassay, as discussed above, includes a substrate upon which the nucleocapsid protein has been immobilized. The variant SARS-CoV-2 spike proteins or fragments thereof include the spike protein variants and fragments thereof containing the mutations as set forth in Tables 1-3 below, including variant RBD fragment comprising amino acids 319-541 of any of SEQ ID NO: 2 or 5. Fragments of the disclosed SARS-CoV-2 variant spike proteins are between 5 and (n−1) consecutive amino acids of a given SARS-CoV-2 spike protein variant. In each instance, a fragment of the spike protein variant will include, within its span of consecutive amino acids, the amino acid mutation associated with the mutant spike protein (which are identified in Table 1, Table 2 and Table 3). The numbering of the amino acid mutation is in relation to the amino acid numbering in SEQ ID NO: 2 or 5 for Tables 1-2. Also within the scope of this disclosure are the spike protein sequences and fragments thereof, including RBD sequences, for various SARS-CoV-2 variants, such as B.1.1.7, B.1.351, P.1, and other known variants of SARS-CoV-2. Sequences for these spike protein variants are available in public databases. Table 3 also provides the amino acid mutations associated with these variants, relative to the wild type-sequence (SEQ ID NO: 2).

TABLE 1
Spike protein variants

| Protein | Amino acid mutation | aa range | AA substitution |
|---|---|---|---|
| S1 | 367 | 319-541 | Any amino acid, preferably F, L, |
| | | | A, V, I, M, Y or W |
| S1 | 342 | 319-541 | Any amino acid, preferably L, A, |
| | | | V, I, M, F, Y or W |
| S1 | 435 | 319-541 | Any amino acid, preferably S, T, |
| | | | N or Q |
| S1 | 458 | 319-541 | Any amino acid, preferably R, H |
| | | | or K |
| S1 | 483 | 319-541 | Any amino acid, preferably A, L, |
| | | | V, I, M, F, Y or W |
| S1 | 354 | 319-541 | Any amino acid, preferably D or E |
| S1/S2 | 683, 685, 986, | 1- | Any amino acid, preferably L, A, |
| | and/or 987 | 1213 | V, I, M, F, Y, G, P or W |

TABLE 2
Preferred Spike protein variants

| Protein | Mutant Type | AA Range | ACE2 Binding |
|---|---|---|---|
| S1 | V367F | aa 319-541 | + |
| S1 | F342L | aa 319-541 | + |
| S1 | A435S | aa 319-541 | + |
| S1 | K458R | aa 319-541 | + |
| S1 | V483A | aa 319-541 | + |
| S1 | N354D | aa 319-541 | + |
| S1/S2 | R683A, R685A, | aa 1-1213 | + |
| | K986P, V987P | | |

TABLE 3
Spike protein variants for SARS-CoV2 variants B.1.351, B.1.1.7, and P.1

| | | | |
|---|---|---|---|
| S1; | 417, 484, and/ | 319- | Any amino acid, preferably N, K, |
| B.1.351 | or 501 | 541 | or Y, more preferably K417N, |
| | | | E484K and/or N501Y, even more |
| | | | preferably K417N, E484K and |
| | | | N501Y |
| S1/S2; | 417, 484, 501, | 1-1213 | Any amino acid, preferably N, |
| B.1.351 | 614 and/or 701 | | K, G, V or Y, more preferably |
| | | | K417N, E484K, N501Y, D614G, |
| | | | and/or A701V, even more |
| | | | preferably K417N, E484K, |
| | | | N501Y, D614G, and A701V |
| S1/S2; | Δ69H, Δ70V, | 1-1213 | Any amino acid, preferably Y, D, |
| B.1.1.7 | Δ144Y, 501, 570, | | G, H, I, A, or H, more preferably |
| | 614, 681, 761, | | N501Y, A570D, D614G, P681H, |
| | 982, and/or 1118 | | T761I, S982A, and/or D1118H, |
| | | | even more preferably N501Y, |
| | | | A570D, D614G, P681H, T761I, |
| | | | S982A, and D1118H |
| S1; P1 | 417, 484, and/ | 319- | Any amino acid, preferably T, K, |
| | or 501 | 541 | Y, more preferably K417T, |
| | | | E484K, and/or N501Y, even more |
| | | | preferably K417T, E484K, and |
| | | | N501Y |
| S1/S2; | 18, 20, 26, 138, | 1-1213 | Any amino acid, preferably F, N, |
| P1 | 190, 417, 484, | | S, Y, T, K, G, or I, more |
| | 501, 614, 655, | | preferably L18F, T20N, P26S, |
| | and/or 1027 | | D138Y, R190S, K417T, E484K, |
| | | | N501Y, D614, H655Y, and/or |
| | | | T1027I, even more preferably |
| | | | L18F, T20N, P26S, D138Y, |
| | | | R190S, K417T, E484K, N501Y, |
| | | | D614, H655Y, and/or T1027I |

With respect to the amino acid mutations at positions 683, 685, 986, and 987, any combination of 2, 3 or 4 amino acid mutations can exist in the spike protein variant or a fragment thereof. In certain embodiments, three or all four amino acid mutations exist within the spike protein variant or fragment thereof. Thus, fragments of the spike protein variants that can be immobilized on a solid support are fragments of S1 and can be between 5 and 685 consecutive amino acids in length, provided that the fragment includes one or more of the amino acid substitutions identified in Table 1 or 2 or 3 (for example, amino acids 13-685 or amino acids 319-541 of SEQ ID NO: 5 or 6). Preferred embodiments provide for spike protein variants, or fragments thereof, that contain a single amino acid substitution as identified in Tables 1 and 2 or the spike variants identified in Table 3. The length or a fragment can include or exclude signal peptides that are processed (for example amino acids 1-12 of SEQ ID NO: 2 or SEQ ID NO: 5). In some embodiments, the amino acid at position 354 of SEQ ID NO: 5 is Asx (aspartic acid or asparagine). Spike protein variants of SEQ ID NO: 5, thus, can contain either aspartic acid or asparagine at position 354 and one or more other mutations identified in Tables 1 and 2 (i.e., at positions 342, 367, 435, 458, 483, 683, 685, 986, and/or 987). Thus, in the case of the spike protein variants, the length can include or exclude the signal peptide of the protein. In various embodiments, the spike protein variant fragments utilized in the practice of the disclosed methods include S1, S2 and RBD variant fragments as disclosed herein. Yet other embodiments provide that at least one of S1, S2 and RBD is a spike protein variant and at least one of S1, S2 and RBD is a fragment of the spike protein containing no amino acid mutations.

In various additional embodiments, each of the disclosed immunoassays can utilize trimerized SARS-CoV-2 spike (S) protein, trimerized variant SARS-CoV-2 spike (S) protein, S1, S2, envelope (E), nucleocapsid (N) and/or membrane (M) protein or fragments thereof (for example, the RBD of the S1 protein) as an analyte and can be immobilized on beads as discussed above. Trimerized SARS-CoV-2 spike (S) protein and trimerized variant SARS-CoV-2 spike (S) protein can be obtained from commercial vendors (for example, Icosagen Technologies, Inc., San Francisco, Calif. or Estonia; ACROBiosystems, Inc., Newark, Del.; or other vendors accessible at the following web site: antibodies-online.com/areas/infectious-disease/covid-19/sars-cov-2-proteins). Alternatively, SARS-CoV-2 spike (S) protein and/or trimerized variant SARS-CoV-2 spike (S) protein or can be constructed from the disclosed spike proteins utilizing trimerization domains. In some embodiments, the furin cleavage (RRAR) site between Spike S1 and S2 can be mutated to prevent cleavage between the S1 and S2 domains.

The disclosure, thus, provides the following non-limiting embodiments:

1. A substrate comprising at least two SARS-CoV-2 proteins or fragments thereof selected from the group consisting of spike (S) and/or fragments thereof, S1 and/or fragments thereof (such as the RBD of S1 or a fragment thereof), S2 and/or fragments thereof, envelope (E) and/or fragments thereof, nucleocapsid (N) and/or fragments thereof and/or membrane (M) protein and/or fragments thereof and combinations thereof, said proteins and/or fragments thereof being immobilized on said substrate.

2. The substrate according to embodiment 1, wherein said substrate is glass, plastic, polystyrene or nitrocellulose.

3. The substrate according to embodiment 1 or 2, wherein said substrate is a particle or a bead.

4. The substrate according to embodiment 3, wherein said substrate is a population of particles or beads comprising two or more separate subpopulations of particles or beads, each subpopulation of particles or beads being distinguishable by a specific detectable parameter and each subpopulation of beads comprising a distinct SARS-CoV-2 protein or fragment thereof immobilized thereon.

5. The substrate according to embodiment 4, wherein said population comprises two or more separate subpopulations of particles or beads selected from:

a) Spike (S) and/or fragments thereof immobilized on a first particle or bead having a first specific detectable physical parameter;

b) S1 and/or fragments thereof immobilized on a second particle or bead having a second specific detectable physical parameter;

c) S2 and/or fragments thereof immobilized on a third particle or bead having a third specific detectable physical parameter;

d) envelope (E) protein and/or fragments thereof immobilized on a fourth particle or bead having a fourth specific detectable physical parameter;

e) nucleocapsid (N) and/or fragments thereof immobilized on a fifth particle or bead having a fifth specific detectable physical parameter; and

f) membrane (M) protein and/or fragments thereof immobilized on a sixth particle or bead having a sixth specific detectable physical parameter.

6. The substrate according to embodiment 5, wherein said population of particles or beads comprises two, three, four, five, or six subpopulations of particles or beads, each subpopulation having a specific detectable physical parameter.

7. The substrate according to any one of embodiments 4-6, wherein the specific detectable physical parameter is a fluorescent dye, fluorophore, luminescent agent, electron-dense reagent, radioisotope or particle size.

8. The substrate according to embodiment 7, wherein the specific detectable parameter is a fluorophore.

9. A method for detecting antibodies in a mammal comprising obtaining a biological sample from the mammal, contacting said biological sample with a substrate comprising a substrate according to any one of embodiments 1-8 and detecting the presence or absence of antibodies bound to SARS-CoV-2 proteins or fragments thereof immobilized on the surface of said substrate.

10. The method according to embodiment 9, wherein the mammal is a human, non-human primate, canine, or feline.

11. A substrate comprising a monoclonal antibody or antigen binding fragment thereof or a polyclonal antibody or antigen binding fragments thereof that specifically bind to a SARS-CoV-2 protein or fragment thereof selected from the group consisting of spike (S) and/or fragments thereof, S1 and/or fragments thereof (such as the RBD of S1 or a fragment thereof), S2 and/or fragments thereof, envelope (E) and/or fragments thereof, nucleocapsid (N) and/or fragments thereof and/or membrane (M) protein and/or fragments thereof, said monoclonal antibody or antigen binding fragment thereof or a polyclonal antibody or antigen binding fragments thereof being immobilized on said substrate.

12. The substrate according to embodiment 11, wherein said substrate is glass, plastic, polystyrene or nitrocellulose.

13. The substrate according to embodiment 11 or 12, wherein said substrate is a particle or a bead.

14. The substrate according to embodiment 13, wherein said substrate is a population of particles or beads comprising two or more separate subpopulations of particles or beads, each subpopulation of particles or beads being distinguishable by a specific detectable parameter and each subpopulation of beads comprising immobilized monoclonal antibodies or antigen binding fragments thereof or polyclonal antibodies or antigen binding fragments thereof that bind to a distinct SARS-CoV-2 protein or fragment thereof

15. The substrate according to embodiment 14, wherein said population comprises two or more separate subpopulations of particles or beads selected from:

a) monoclonal antibodies or antigen binding fragments thereof or polyclonal antibodies or antigen binding fragments thereof that specifically bind spike (S) and/or fragments thereof, said monoclonal antibodies or antigen binding fragments thereof or polyclonal antibodies or antigen binding fragments thereof being immobilized on a first particle or bead having a first specific detectable physical parameter;

b) monoclonal antibodies or antigen binding fragments thereof or polyclonal antibodies or antigen binding fragments thereof that specifically bind S1 and/or fragments thereof (such as the RBD of S1 or a fragment thereof), said monoclonal antibodies or antigen binding fragments thereof or polyclonal antibodies or antigen binding fragments thereof being immobilized on a second particle or bead having a second specific detectable physical parameter;

c) monoclonal antibodies or antigen binding fragments thereof or polyclonal antibodies or antigen binding fragments thereof that specifically bind S2 and/or fragments thereof, said monoclonal antibodies or antigen binding fragments thereof or polyclonal antibodies or antigen binding fragments thereof being immobilized on a third particle or bead having a third specific detectable physical parameter;

d) monoclonal antibodies or antigen binding fragments thereof or polyclonal antibodies or antigen binding fragments thereof that specifically bind envelope (E) protein and/or fragments thereof, said monoclonal antibodies or antigen binding fragments thereof or polyclonal antibodies or antigen binding fragments thereof being immobilized on a fourth particle or bead having a fourth specific detectable physical parameter;

e) monoclonal antibodies or antigen binding fragments thereof or polyclonal antibodies or antigen binding fragments thereof that specifically bind nucleocapsid (N) and/or fragments thereof, said monoclonal antibodies or antigen binding fragments thereof or polyclonal antibodies or antigen binding fragments thereof being immobilized on a fifth particle or bead having a fifth specific detectable physical parameter; and

f) monoclonal antibodies or antigen binding fragments thereof or polyclonal antibodies or antigen binding fragments thereof that specifically bind membrane (M) protein and/or fragments thereof, said monoclonal antibodies or antigen binding fragments thereof or polyclonal antibodies or antigen binding fragments thereof being immobilized on an sixth particle or bead having an sixth specific detectable physical parameter.

16. The substrate according to embodiment 15, wherein said population of particles or beads comprises two, three, four, five, or six subpopulations of particles or beads, each subpopulation having a specific detectable physical parameter.

17. The substrate according to any one of embodiments 14-16, wherein the specific detectable physical parameter is a fluorescent dye (fluorophore), luminescent agent, electron-dense reagent, radioisotope or particle size.

18. The substrate according to embodiment 17, wherein the specific detectable parameter is a fluorophore.

19. A method for detecting SARS-CoV-2 infection in a mammal comprising obtaining a biological sample from the mammal, contacting said biological sample with a substrate according to any one of embodiments 11-18 and detecting the presence or absence of SARS-CoV-2 proteins or fragments thereof in said biological sample that are bound to the antibodies immobilized on the surface of said substrate.

20. The method according to embodiment 19, wherein the mammal is a human, non-human primate, canine, or feline.

21. The method according to embodiment 20, wherein the mammal is a human.

22. The method according to any one of embodiments 9-10, wherein said detection comprises contacting the substrate contacted with said biological sample with anti-immunoglobulin (anti-Ig) antibodies that are detectably labeled with a fluorescent dye (fluorophore), luminescent agent, electron-dense reagent, or radioisotope.

23. The method according to embodiment 22, wherein said anti-Ig antibodies are detectably labeled with a fluorophore specific for each class of immunoglobulin and the sample fluorescence intensity is compared to a set of standards or calibrators to generate a qualitative, semi-quantitative or quantitative result.

24. The method according to any one of embodiments 9-10, wherein said detection comprises contacting the substrate contacted with said biological sample with anti-Ig antibodies that are biotinylated and contacting said biotinylated antibodies with a biotin-binding ligand that is detectably labeled.

25. The method according to embodiment 24, the sample fluorescence intensity is compared to a set of standards or calibrators to generate a qualitative, semi-quantitative or quantitative result of total Ig bound to said substrate.

26. The method according to any one of embodiments 19-21, wherein said detection comprises contacting the substrate contacted with said biological sample with biotinylated antibody specific for each distinct SARS-CoV-2 protein or fragment thereof and contacting said biotinylated antibodies bound to SARS-CoV-2 protein or fragment thereof that are bound to said substrate with a biotin-binding ligand that is detectably labeled and determining fluorescence intensity of the substrate.

27. The method according to embodiment 26, the sample fluorescence intensity is compared to a set of standards or calibrators to generate a qualitative, semi-quantitative or quantitative result of total Ig bound to said substrate.

28. The method according to any one of embodiments 9-10 or 22-25, said method comprising contacting the biological sample with a substrate according to any one of embodiments 11-18 and detecting the presence or absence of SARS-CoV-2 proteins or fragments thereof in said biological sample that are bound to the antibodies immobilized on the surface of said substrate.

29. The method according to any one of embodiments 19-21 or 26-27, said method comprising contacting the biological sample with a substrate according to any one of embodiments 1-8 and detecting the presence or absence of antibodies bound to SARS-CoV-2 proteins or fragments thereof immobilized on the surface of said substrate.

30. A substrate comprising a population of particles or beads comprising two or more separate subpopulations of particles or beads, each subpopulation of particles or beads being distinguishable by a specific detectable parameter and each subpopulation of beads comprising immobilized monoclonal antibodies or antigen binding fragments thereof or polyclonal antibodies or antigen binding fragments thereof that bind to a distinct SARS-CoV-2 protein or fragment thereof or a cytokine selected from selected from IL-1beta, IFN-γ, IFNγ-induced protein 10 (IP-10), monocyte chemoattractant protein 1 (MCP-1), IL-4, IL-10, IL-2R, IL-6, granulocyte colony-stimulating factor (G-CSF), macrophage inflammatory protein-1A, and TNF-α.

31. The substrate according to embodiment 30, wherein said population comprises two or more separate subpopulations of particles or beads selected from:

a) monoclonal antibodies or antigen binding fragments thereof or polyclonal antibodies or antigen binding fragments thereof that specifically bind spike (S) and/or fragments thereof, said monoclonal antibodies or antigen binding fragments thereof or polyclonal antibodies or antigen binding fragments thereof being immobilized on a first particle or bead having a first specific detectable physical parameter;

b) monoclonal antibodies or antigen binding fragments thereof or polyclonal antibodies or antigen binding fragments thereof that specifically bind S1 and/or fragments thereof (such as the RBD of S1 or a fragment thereof), said monoclonal antibodies or antigen binding fragments thereof or polyclonal antibodies or antigen binding fragments thereof being immobilized on a second particle or bead having a second specific detectable physical parameter;

c) monoclonal antibodies or antigen binding fragments thereof or polyclonal antibodies or antigen binding fragments thereof that specifically bind S2 and/or fragments thereof, said monoclonal antibodies or antigen binding fragments thereof or polyclonal antibodies or antigen binding fragments thereof being immobilized on a third particle or bead having a third specific detectable physical parameter;

d) monoclonal antibodies or antigen binding fragments thereof or polyclonal antibodies or antigen binding fragments thereof that specifically bind envelope (E) protein and/or fragments thereof, said monoclonal antibodies or antigen binding fragments thereof or polyclonal antibodies or antigen binding fragments thereof being immobilized on a fourth particle or bead having a fourth specific detectable physical parameter;

e) monoclonal antibodies or antigen binding fragments thereof or polyclonal antibodies or antigen binding fragments thereof that specifically bind nucleocapsid (N) and/or fragments thereof, said monoclonal antibodies or antigen binding fragments thereof or polyclonal antibodies or antigen binding fragments thereof being immobilized on a fifth particle or bead having a fifth specific detectable physical parameter;

f) monoclonal antibodies or antigen binding fragments thereof or polyclonal antibodies or antigen binding fragments thereof that specifically bind membrane (M) protein and/or fragments thereof, said monoclonal antibodies or antigen binding fragments thereof or polyclonal antibodies or antigen binding fragments thereof being immobilized on a sixth particle or bead having a sixth specific detectable physical parameter;

g) monoclonal antibodies or antigen binding fragments thereof or polyclonal antibodies or antigen binding fragments thereof that specifically bind IL-1beta, said monoclonal antibodies or antigen binding fragments thereof or polyclonal antibodies or antigen binding fragments thereof being immobilized on a seventh particle or bead having a seventh specific detectable physical parameter;

h) monoclonal antibodies or antigen binding fragments thereof or polyclonal antibodies or antigen binding fragments thereof that specifically bind IFN-γ, said monoclonal antibodies or antigen binding fragments thereof or polyclonal antibodies or antigen binding fragments thereof being immobilized on an eighth or bead having an eighth specific detectable physical parameter;

i) monoclonal antibodies or antigen binding fragments thereof or polyclonal antibodies or antigen binding fragments thereof that specifically bind IFNγ-induced protein 10 (IP-10), said monoclonal antibodies or antigen binding fragments thereof or polyclonal antibodies or antigen binding fragments thereof being immobilized on a ninth particle or bead having a ninth specific detectable physical parameter;

j) monoclonal antibodies or antigen binding fragments thereof or polyclonal antibodies or antigen binding fragments thereof that specifically bind monocyte chemoattractant protein 1 (MCP-1), said monoclonal antibodies or antigen binding fragments thereof or polyclonal antibodies or antigen binding fragments thereof being immobilized on a tenth particle or bead having a tenth specific detectable physical parameter;

k) monoclonal antibodies or antigen binding fragments thereof or polyclonal antibodies or antigen binding fragments thereof that specifically bind IL-4, said monoclonal antibodies or antigen binding fragments thereof or polyclonal antibodies or antigen binding fragments thereof being immobilized on an eleventh particle or bead having an eleventh specific detectable physical parameter;

l) monoclonal antibodies or antigen binding fragments thereof or polyclonal antibodies or antigen binding fragments thereof that specifically bind IL-10, said monoclonal antibodies or antigen binding fragments thereof or polyclonal antibodies or antigen binding fragments thereof being immobilized on a twelfth particle or bead having a twelfth specific detectable physical parameter;

m) monoclonal antibodies or antigen binding fragments thereof or polyclonal antibodies or antigen binding fragments thereof that specifically bind IL-2R, said monoclonal antibodies or antigen binding fragments thereof or polyclonal antibodies or antigen binding fragments thereof being immobilized on a thirteenth particle or bead having a thirteenth specific detectable physical parameter;

n) monoclonal antibodies or antigen binding fragments thereof or polyclonal antibodies or antigen binding fragments thereof that specifically bind IL-6, said monoclonal antibodies or antigen binding fragments thereof or polyclonal antibodies or antigen binding fragments thereof being immobilized on a fourteenth particle or bead having a fourteenth specific detectable physical parameter;

o) monoclonal antibodies or antigen binding fragments thereof or polyclonal antibodies or antigen binding fragments thereof that specifically bind granulocyte colony-stimulating factor (G-CSF), said monoclonal antibodies or antigen binding fragments thereof or polyclonal antibodies or antigen binding fragments thereof being immobilized on a fifteenth particle or bead having a fifteenth specific detectable physical parameter;

p) monoclonal antibodies or antigen binding fragments thereof or polyclonal antibodies or antigen binding fragments thereof that specifically bind macrophage inflammatory protein-1A, said monoclonal antibodies or antigen binding fragments thereof or polyclonal antibodies or antigen binding fragments thereof being immobilized on a sixteenth particle or bead having a sixteenth specific detectable physical parameter; and

q) monoclonal antibodies or antigen binding fragments thereof or polyclonal antibodies or antigen binding fragments thereof that specifically bind TNF-α, said monoclonal antibodies or antigen binding fragments thereof or polyclonal antibodies or antigen binding fragments thereof being immobilized on a seventeenth particle or bead having a seventeenth specific detectable physical parameter.

32. The substrate according to embodiment 31, wherein said population of particles or beads comprises 2, 3, 4, 5, 6, 7, 8, 9, 10, 11, 12, 13, 14, 15, 16 or 17 subpopulations of particles or beads, each subpopulation having a specific detectable physical parameter.

33. The substrate according to any one of embodiments 30-32, wherein the specific detectable physical parameter is a fluorescent dye (fluorophore), luminescent agent, electron-dense reagent, radioisotope or particle size.

34. The substrate according to embodiment 33, wherein the specific detectable parameter is a fluorophore.

35. The substrate according to any one of embodiments 30-34, wherein said particle or bead is glass, plastic, polystyrene or nitrocellulose.

36. A method for detecting SARS-CoV-2 infection in a mammal comprising obtaining a biological sample from the mammal, contacting said biological sample with a substrate according to any one of embodiments 30-35 and detecting the presence or absence of SARS-CoV-2 proteins or fragments thereof and, optionally, cytokines selected from IL-1beta, IFN-γ, IFNγ-induced protein 10 (IP-10), monocyte chemoattractant protein 1 (MCP-1), IL-4, IL-10, IL-2R, IL-6, granulocyte colony-stimulating factor (G-CSF), macrophage inflammatory protein-1A, and TNF-α in said biological sample that are bound to the antibodies immobilized on the surface of said substrate.

37. The method according to embodiment 36, wherein the mammal is a human, non-human primate, canine, or feline.

38. The method according to embodiment 37, wherein the mammal is a human.

39. The method according to any one of embodiments 36-38, wherein said detection comprises contacting the substrate contacted with said biological sample with biotinylated antibody specific for each distinct SARS-CoV-2 protein or fragment thereof and, optionally, biotinylated antibody specific for each selected cytokine and contacting said biotinylated antibodies bound to SARS-CoV-2 protein or fragment thereof and, when assayed, said biotinylated antibody specific for each selected cytokine that are bound to said substrate with a biotin-binding ligand that is detectably labeled and determining the fluorescence intensity for each subpopulation of beads.

40. The method according to embodiment 39, the sample fluorescence intensity is compared to a set of standards or calibrators to generate a qualitative, semi-quantitative or quantitative result.

41. A substrate comprising a population of particles or beads comprising two or more separate subpopulations of particles or beads, each subpopulation of particles or beads being distinguishable by a specific detectable parameter and each subpopulation of beads comprising a distinct SARS-CoV-2 protein or fragment thereof or antibodies specific for a cytokine selected from IL-1beta, IFN-γ, IFNγ-induced protein 10 (IP-10), monocyte chemoattractant protein 1 (MCP-1), IL-4, IL-10, IL-2R, IL-6, granulocyte colony-stimulating factor (G-CSF), macrophage inflammatory protein-1A, and TNF-α immobilized thereon.

42. The substrate according to embodiment 41, wherein said population comprises two or more separate subpopulations of particles or beads selected from:

a) Spike (S) and/or fragments thereof immobilized on a first particle or bead having a first specific detectable physical parameter;

b) S1 and/or fragments thereof (such as the RBD of S1 or a fragment thereof) immobilized on a second particle or bead having a second specific detectable physical parameter;

c) S2 and/or fragments thereof immobilized on a third particle or bead having a third specific detectable physical parameter;

d) envelope (E) protein and/or fragments thereof immobilized on a fourth particle or bead having a fourth specific detectable physical parameter;

e) nucleocapsid (N) and/or fragments thereof immobilized on a fifth particle or bead having a fifth specific detectable physical parameter;

f) membrane (M) protein and/or fragments thereof immobilized on a sixth particle or bead having a sixth specific detectable physical parameter;

g) monoclonal antibodies or antigen binding fragments thereof or polyclonal antibodies or antigen binding fragments thereof that specifically bind IL-1beta, said monoclonal antibodies or antigen binding fragments thereof or polyclonal antibodies or antigen binding fragments thereof being immobilized on a seventh particle or bead having a seventh specific detectable physical parameter;

h) monoclonal antibodies or antigen binding fragments thereof or polyclonal antibodies or antigen binding fragments thereof that specifically bind IFN-γ, said monoclonal antibodies or antigen binding fragments thereof or polyclonal antibodies or antigen binding fragments thereof being immobilized on an eighth or bead having an eighth specific detectable physical parameter;

i) monoclonal antibodies or antigen binding fragments thereof or polyclonal antibodies or antigen binding fragments thereof that specifically bind IFNγ-induced protein 10 (IP-10), said monoclonal antibodies or antigen binding fragments thereof or polyclonal antibodies or antigen binding fragments thereof being immobilized on a ninth particle or bead having a ninth specific detectable physical parameter;

j) monoclonal antibodies or antigen binding fragments thereof or polyclonal antibodies or antigen binding fragments thereof that specifically bind monocyte chemoattractant protein 1 (MCP-1), said monoclonal antibodies or antigen binding fragments thereof or polyclonal antibodies or antigen binding fragments thereof being immobilized on a tenth particle or bead having a tenth specific detectable physical parameter;

k) monoclonal antibodies or antigen binding fragments thereof or polyclonal antibodies or antigen binding fragments thereof that specifically bind IL-4, said monoclonal antibodies or antigen binding fragments thereof or polyclonal antibodies or antigen binding fragments thereof being immobilized on an eleventh particle or bead having an eleventh specific detectable physical parameter;

l) monoclonal antibodies or antigen binding fragments thereof or polyclonal antibodies or antigen binding fragments thereof that specifically bind IL-10, said monoclonal antibodies or antigen binding fragments thereof or polyclonal antibodies or antigen binding fragments thereof being immobilized on a twelfth particle or bead having a twelfth specific detectable physical parameter;

m) monoclonal antibodies or antigen binding fragments thereof or polyclonal antibodies or antigen binding fragments thereof that specifically bind IL-2R, said monoclonal antibodies or antigen binding fragments thereof or polyclonal antibodies or antigen binding fragments thereof being immobilized on a thirteenth particle or bead having a thirteenth specific detectable physical parameter;

n) monoclonal antibodies or antigen binding fragments thereof or polyclonal antibodies or antigen binding fragments thereof that specifically bind IL-6, said monoclonal antibodies or antigen binding fragments thereof or polyclonal antibodies or antigen binding fragments thereof being immobilized on a fourteenth particle or bead having a fourteenth specific detectable physical parameter;

o) monoclonal antibodies or antigen binding fragments thereof or polyclonal antibodies or antigen binding fragments thereof that specifically bind granulocyte colony-stimulating factor (G-CSF), said monoclonal antibodies or antigen binding fragments thereof or polyclonal antibodies or antigen binding fragments thereof being immobilized on a fifteenth particle or bead having a fifteenth specific detectable physical parameter;

p) monoclonal antibodies or antigen binding fragments thereof or polyclonal antibodies or antigen binding fragments thereof that specifically bind macrophage inflammatory protein-1A, said monoclonal antibodies or antigen binding fragments thereof or polyclonal antibodies or antigen binding fragments thereof being immobilized on a sixteenth particle or bead having a sixteenth specific detectable physical parameter; and

q) monoclonal antibodies or antigen binding fragments thereof or polyclonal antibodies or antigen binding fragments thereof that specifically bind TNF-α, said monoclonal antibodies or antigen binding fragments thereof or polyclonal antibodies or antigen binding fragments thereof being immobilized on a seventeenth particle or bead having a seventeenth specific detectable physical parameter.

43. The substrate according to embodiment 42, wherein said population of particles or beads comprises 2, 3, 4, 5, 6, 7, 8, 9, 10, 11, 12, 13, 14, 15, 16 or 17 subpopulations of particles or beads, each subpopulation having a specific detectable physical parameter.

44. The substrate according to any one of embodiments 41-43, wherein the specific detectable physical parameter is a fluorescent dye (fluorophore), luminescent agent, electron-dense reagent, radioisotope or particle size.

45. The substrate according to embodiment 44, wherein the specific detectable parameter is a fluorophore.

46. The substrate according to any one of embodiments 41-45, wherein said particle or bead is glass, plastic, polystyrene or nitrocellulose.

47. A method for detecting SARS-CoV2 antibodies and, optionally, cytokines in a mammal comprising obtaining a biological sample from the mammal, contacting said biological sample with a substrate according to any one of embodiments 41-46 and detecting the presence or absence of antibodies bound to SARS-CoV-2 proteins or fragments thereof and, optionally, the presence or absence of cytokines.

48. The method according to embodiment 47, wherein the mammal is a human, non-human primate, canine, or feline.

49. The method according to any one of embodiments 47-48, wherein said detection comprises contacting the substrate contacted with said biological sample with anti-immunoglobulin (anti-Ig) antibodies and, optionally, cytokine specific antibodies that are detectably labeled with a fluorescent dye (fluorophore), luminescent agent, electron-dense reagent, or radioisotope.

50. The method according to embodiment 49, wherein said anti-Ig antibodies are detectably labeled with a fluorophore specific for each class of immunoglobulin and a fluorophore specific for each cytokine, when assayed, and the sample fluorescence intensity is compared to a set of standards or calibrators to generate a qualitative, semi-quantitative or quantitative result.

51. The method according to any one of embodiments 47-48, wherein said detection comprises contacting the substrate contacted with said biological sample with anti-Ig antibodies and cytokine specific antibodies, when assayed, that are biotinylated and contacting said biotinylated antibodies with a biotin-binding ligand that is detectably labeled with a fluorophore and determining the fluorescence intensity for each subpopulation of beads.

52. The method according to embodiment 51, the sample fluorescence intensity is compared to a set of standards or calibrators to generate a qualitative, semi-quantitative or quantitative result of total Ig bound to said substrate.

53. The method according to any one of embodiments 36-40 or 47-52, wherein said method assays for the presence and/or quantity of one or more of said cytokines.

54. The method according to embodiment 53, wherein said one or more cytokine is IL-6 and/or IL-2R.

55. The method according to embodiment 53, wherein said one or more cytokine is selected from IL-6, IL-2R, granulocyte colony-stimulating factor, IP-10, MCP-1, macrophage inflammatory protein-1A, TNF-α, and combinations thereof.

56. The substrate according to any one of embodiments 1-8, wherein said population of four or more separate subpopulations of particles or beads comprises:

a) the spike protein receptor binding domain (RBD) immobilized on a first particle or bead having a first specific detectable physical parameter;

b) S1 immobilized on a second particle or bead having a second specific detectable physical parameter;

c) S2 immobilized on a third particle or bead having a third specific detectable physical parameter; and

d) nucleocapsid (N) immobilized on a fourth particle or bead having a fourth specific detectable physical parameter.

57. A method of differentiating mammals infected with SARS-CoV-2 from mammals vaccinated with a spike protein vaccine comprising obtaining a biological sample from the mammal, contacting said biological sample with a substrate and detecting the presence or absence of antibodies bound to SARS-CoV-2 nucleocapsid immobilized on a substrate, optionally, the presence or absence of cytokines,

wherein said substrate comprises a population four or more separate subpopulations of particles or beads selected from:

a) the spike protein receptor binding domain (RBD) immobilized on a first particle or bead having a first specific detectable physical parameter;

b) S1 immobilized on a second particle or bead having a second specific detectable physical parameter;

c) S2 immobilized on a third particle or bead having a third specific detectable physical parameter; and

d) nucleocapsid (N) immobilized on a fourth particle or bead having a fourth specific detectable physical parameter.

58. The method according to embodiment 57, wherein the mammal is a human, non-human primate, canine, or feline.

59. The method according to any one of embodiments 57-58, wherein said detection comprises contacting the substrate contacted with said biological sample with detectably labeled anti-immunoglobulin (anti-Ig) antibodies.

60. The method according to embodiment 59, wherein said method quantifies antibody amounts in the biological sample by measuring the amount of antibody captured by each subpopulation of beads by the intensity of the attached labeled anti-Ig on each subpopulation of beads.

61. The method according to embodiment 60, wherein the labelled anti-Ig is labeled with a fluorescent label, the identity of each subpopulation of particles or beads is determined by the fluorescence signature of the dyed beads, and the amount of antibody captured by the antigen is determined by the fluorescence intensity of the attached labeled anti-Ig.

62. The method according to embodiment 61, wherein the sample fluorescence intensity is compared to a set of standards or calibrators to generate a qualitative, semi-quantitative or quantitative result of total Ig bound to said substrate.

63. The method according to any one of embodiments 57-58, wherein said detection comprises contacting the substrate contacted with said biological sample with anti-Ig antibodies that are biotinylated and contacting said biotinylated antibodies with a biotin-binding ligand that is detectably labeled with a fluorophore and determining the fluorescence intensity for each subpopulation of beads.

64. The method according to embodiment 63, wherein the sample fluorescence intensity is compared to a set of standards or calibrators to generate a qualitative, semi-quantitative or quantitative result of total Ig bound to said substrate.

65. A substrate comprising at least two SARS-CoV-2 proteins or fragments thereof selected from the group consisting of spike protein (S) and/or fragments thereof, S1 and/or fragments thereof, RBD or a fragment thereof, S2 and/or fragments thereof, spike protein (S) and/or fragments thereof, S1 protein variants and/or fragments thereof, RBD variants or a fragment thereof, S2 protein variants and/or fragments thereof, envelope (E) and/or fragments thereof, nucleocapsid (N) and/or fragments thereof and/or membrane (M) protein and/or fragments thereof and combinations thereof, said proteins and/or fragments thereof being immobilized on said substrate, wherein:

said substrate is a population of particles or beads comprising two or more separate subpopulations of particles or beads, each subpopulation of particles or beads being distinguishable by a specific detectable parameter;

each subpopulation of particles or beads comprises a distinct SARS-CoV-2 protein or fragment thereof immobilized thereon; and

at least one subpopulation of particles or beads comprises a SARS-CoV-2 protein variant selected from the group consising of spike protein variants and/or fragments thereof, S1 protein variants and/or fragments thereof, RBD variants or a fragment thereof, S2 protein variants and/or fragments thereof.

66. The substrate according to embodiment 65, wherein said substrate is glass, plastic, polystyrene or nitrocellulose.

67. The substrate according to embodiment 65 or 66, wherein said population of particles or beads comprises at least two or three subpopulations of particles or beads, each subpopulation comprising a distinct SARS-CoV-2 protein variant immobilized thereon.

68. The substrate according to embodiment 65 or 66, wherein said substrate is a population of particles or beads comprising two or more separate subpopulations of particles or beads, each subpopulation of particles or beads being distinguishable by a specific detectable parameter and each subpopulation of beads comprising a distinct SARS-CoV-2 protein or fragment thereof immobilized thereon and at least one subpopulation of particles or beads comprising a SARS-CoV-2 protein variant selected from the group consising of spike protein variants and/or fragments thereof, S1 protein variants and/or fragments thereof, RBD variants or a fragment thereof, S2 protein variants and/or fragments thereof.

69. The substrate according to embodiments 65-68, wherein said population comprises two or more separate subpopulations of particles or beads selected from:

a) a SARS-CoV-2 spike protein variant or fragment thereof containing the mutation V367X immobilized on a first particle or bead having a first specific detectable physical parameter, where X is any amino acid or X is F;

b) a SARS-CoV-2 spike protein variant or fragment thereof containing the mutation F342X immobilized on a second particle or bead having a second specific detectable physical parameter, where X is any amino acid or X is L;

c) a SARS-CoV-2 spike protein variant or fragment thereof containing the mutation A435X immobilized on a third particle or bead having a third specific detectable physical parameter, where X is any amino acid or X is S;

d) a SARS-CoV-2 spike protein variant or fragment thereof containing the mutation K458X immobilized on a fourth particle or bead having a fourth specific detectable physical parameter, where X is any amino acid or X is R;

e) a SARS-CoV-2 spike protein variant or fragment thereof containing the mutation V483X immobilized on a fifth particle or bead having a fifth specific detectable physical parameter, where X is any amino acid or X is A;

f) a SARS-CoV-2 spike protein variant or fragment thereof containing the mutation V483X immobilized on a sixth particle or bead having a sixth specific detectable physical parameter, where X is any amino acid or X is A;

g) a SARS-CoV-2 spike protein variant or fragment thereof containing the mutation N354X immobilized on a seventh particle or bead having a seventh specific detectable physical parameter, where X is any amino acid or X is D;

h) a SARS-CoV-2 spike protein variant or fragment thereof containing the mutations R683X1 and/or R685X2 immobilized on an eighth particle or bead having an eighth specific detectable physical parameter, where X1 and X2 are any amino acid or X1 and X2 are A;

i) a SARS-CoV-2 spike protein variant or fragment thereof containing the mutations K986X3 and/or V987X4 immobilized on a ninth particle or bead having a ninth specific detectable physical parameter, where X3 and X4 are any amino acid or X3 and X4 are P;

j) a SARS-CoV-2 spike protein variant or fragment thereof containing the mutations R683X1, R685X2, K986X3, and V987X4 immobilized on a tenth particle or bead having a tenth specific detectable physical parameter, where X1 is any amino acid or X1 is A; X2 is any amino acid or X2 is A; X3 is any amino acid or X3 is P; and X4 is any amino acid or X4 is P;

k) a SARS-CoV-2 spike protein (SEQ ID NO: 2) or fragment thereof immobilized on an eleventh particle or bead having an eleventh specific detectable physical parameter;

l) a SARS-CoV-2 spike protein or fragment thereof derived from SARS-CoV-2 variant B.1.1.7 immobilized on a twelfth particle or bead having a twelfth specific detectable physical parameter;

m) a SARS-CoV-2 spike protein or fragment thereof derived from SARS-CoV-2 variant B.1.351 immobilized on a thirteenth particle or bead having a thirteenth eleventh specific detectable physical parameter; and/or

n) a SARS-CoV-2 spike protein or fragment thereof derived from SARS-CoV-2 variant P.1 immobilized on a fourteenth particle or bead having a fourteenth specific detectable physical parameter,

wherein the mutations are relative to the amino acid numbering of SEQ ID NO: 2.

70. The substrate according to embodiment 70, wherein the population of particles or beads comprises subpopulations of particles or beads that contain the S1, S2 and RBD fragments of said variant spike proteins.

71. The substrate according to embodiment 69 or 70, wherein the population of particles or beads comprises at least one subpopulation of particles or beads that contain wild-type S1, S2 or RBD fragments of the spike protein and at least one subpopulation of particles or beads that contain the S1, S2 and RBD fragments of said variant spike proteins.

72. The substrate according to embodiment 71, wherein a combined population of spike protein beads is formed that comprises one or two of said subpopulations of particles or beads that comprise wild-type S1, S2 or RBD fragments of the spike protein and one or two subpopulations of particles or beads that comprise the S1, S2 and RBD fragments of said variant spike proteins, provided that the combined population of spike protein beads is composed of a subpopulation of particles or beads that comprises a wild-type S1 protein or S1 variant, a subpopulation of particles or beads that comprises a wild-type S2 protein or S2 variant, and a subpopulation of particles or beads that comprises a wild-type RBD protein or RBD variant.

73. The substrate according to any one of embodiments 69-72, wherein said population of particles or beads comprises two, three, four, five, six, seven, eight, nine, ten or more subpopulations of particles or beads, each subpopulation having a specific detectable physical parameter.

74. The substrate according to any one of embodiments 68-73, wherein the specific detectable physical parameter is a fluorescent dye, fluorophore, luminescent agent, electron-dense reagent, radioisotope or particle size.

75. The substrate according to embodiment 74, wherein the specific detectable parameter is a fluorophore.

76. The substrate according to any one of embodiments 65-68, wherein said population of particles or beads comprises four or more separate subpopulations of particles or beads comprises:

a) a variant spike protein receptor binding domain (RBD) immobilized on a first particle or bead having a first specific detectable physical parameter;

b) a variant S1 immobilized on a second particle or bead having a second specific detectable physical parameter;

c) a variant S2 immobilized on a third particle or bead having a third specific detectable physical parameter; and

d) nucleocapsid (N) immobilized on a fourth particle or bead having a fourth specific detectable physical parameter.

77. A method for detecting antibodies in a mammal comprising obtaining a biological sample from the mammal, contacting said biological sample with a substrate comprising a substrate according to any one of embodiments 65-76 and detecting the presence or absence of antibodies bound to SARS-CoV-2 proteins or fragments thereof immobilized on the surface of said substrate.

78. The method according to embodiment 77, wherein the mammal is a human, non-human primate, canine, or feline.

79. A method of differentiating mammals infected with SARS-CoV-2 from mammals vaccinated with a spike protein vaccine comprising obtaining a biological sample from the mammal, contacting said biological sample with a substrate and detecting the presence or absence of antibodies bound to SARS-CoV-2 nucleocapsid immobilized on a substrate, optionally, the presence or absence of cytokines,

wherein said substrate comprises a population four or more separate subpopulations of particles or beads selected from:

a) the spike protein receptor binding domain (RBD) or RBD variant immobilized on a first particle or bead having a first specific detectable physical parameter;

b) S1 or a variant thereof immobilized on a second particle or bead having a second specific detectable physical parameter;

c) S2 or a variant thereof immobilized on a third particle or bead having a third specific detectable physical parameter; and d) nucleocapsid (N) immobilized on a fourth particle or bead having a fourth specific detectable physical parameter, provided that at least one subpopulation of particles or beads provides a RBD variant, S1 variant or S2 variant.

80. The method according to any one of embodiments 77-79, wherein the mammal is a human, non-human primate, canine, or feline.

81. The method according to any one of embodiments 77-80, wherein said detection comprises contacting the substrate contacted with said biological sample with detectably labeled anti-immunoglobulin (anti-Ig) antibodies.

82. The method according to embodiment 81, wherein said method quantifies antibody amounts in the biological sample by measuring the amount of antibody captured by each subpopulation of beads by the intensity of the attached labeled anti-Ig on each subpopulation of beads.

83. The method according to embodiment 82, wherein the labelled anti-Ig is labeled with a fluorescent label, the identity of each subpopulation of particles or beads is determined by the fluorescence signature of the dyed beads, and the amount of antibody captured by the antigen is determined by the fluorescence intensity of the attached labeled anti-Ig.

84. The method according to embodiment 83, wherein the sample fluorescence intensity is compared to a set of standards or calibrators to generate a qualitative, semi-quantitative or quantitative result of total Ig bound to said substrate.

85. The method according to any one of embodiments 77-80, wherein said detection comprises contacting the substrate contacted with said biological sample with anti-Ig antibodies that are biotinylated and contacting said biotinylated antibodies with a biotin-binding ligand that is detectably labeled with a fluorophore and determining the fluorescence intensity for each subpopulation of beads.

86. The method according to embodiment 85, wherein the sample fluorescence intensity is compared to a set of standards or calibrators to generate a qualitative, semi-quantitative or quantitative result of total Ig bound to said substrate.

### EXAMPLES

### Example 1

### Measurement of IgM/IgA Serology

Fluorescently labeled beads, to which a fragment of S1 comprising amino acids 13-685, a S1 fragment comprising the RBD, N protein and S2 antigen is immobilized, is contacted with a patient sample, e.g., a biological sample from an individual suspected of having been exposed to SARS-CoV-2 virus along with a sample diluent. Sample is diluted with a diluent containing anti-human IgG antibodies so as to block the binding of human IgG to the S1 protein fragment thereby increasing the detection of IgA or IgM. After incubation, unbound sample is washed away and labeled anti-human IgA and anti-human IgM antibodies are added to allow for detection of antibodies from the sample bound to the solid support. The reaction can be incubated and washed prior to detection using a BioPlex 2200, Bio-Plex 200 or Luminex LX-200 platform. The amount of antibody captured by the SARS-CoV-2 antigens is determined by the fluorescence intensity of the attached labeled anti-IgA and anti-IgM and the sample fluorescence intensity is compared to a set of standards or calibrators to generate a qualitative, semi-quantitative or quantitative result.

### Example 2

### Measurement of IgG Serology

Fluorescently labeled beads, to which a S1 fragment comprising the RBD, N protein and S2 antigen are immobilized, is contacted with a patient sample, e.g., a biological sample from an individual suspected of having been exposed to SARS-CoV-2 virus along with a sample diluent. After incubation, unbound sample is washed away and labeled anti-human IgG antibodies are added to allow for detection of antibodies from the sample bound to the solid support. The reaction can be incubated and washed prior to detection using a BioPlex 2200, Bio-Plex 200 or Luminex LX-200 platform. The amount of antibody captured by the SARS-CoV-2 antigens is determined by the fluorescence intensity of the attached labeled anti-IgG antibodies and the sample fluorescence intensity is compared to a set of standards or calibrators to generate a qualitative, semi-quantitative or quantitative result.

### Example 3

### Measurement of Total Serological Response

A population of fluorescently labeled beads comprising a plurality of subpopulations of fluorescently labeled beads to which the RBD of S1 (bead subpopulation 1), N protein (bead subpopulation 2) and S2 antigen (bead subpopulation 3) are immobilized is contacted with a patient sample, e.g., a biological sample from an individual suspected of having been exposed to SARS-CoV-2 virus along with a sample diluent. After incubation unbound sample was washed away and biotinylated protein specific for each immunoglobulin class (for example, biotinylated antibody specific for each class of antibody (IgM, IgA, and IgG) or a universal detection reporter antibody) is added to the washed beads and incubated. Unbound biotinylated protein is then washed away and labeled streptavidin-PE (Phycoerythrin) is added to allow for detection of total bound antibodies. The reaction is incubated and then washed prior to detection using a BioPlex 2200, Bioplex 200 or Luminex LX-200 platform. The amount of total antibody captured by the substrate is determined by the fluorescence intensity of the labeled Streptavidin bound to biotinylated antibody. The amount of total antibody captured by each subpopulation of beads can also be determined by measuring the fluorescence intensity of the labeled Streptavidin bound to biotinylated antibody associated with each subpopulation of beads. The sample fluorescence intensity is compared to the fluorescence intensity of a set of standards or calibrators to generate a qualitative, semi-quantitative or quantitative result.

### Example 4

### Multiplex Detection of SARS-CoV-2 Antigens and Antibodies

A population of fluorescently labeled beads comprising: 1) one or more subpopulations of fluorescently labeled beads to which monoclonal and/or polyclonal antibodies that bind SARS-CoV-2 RBD, N protein and S2 antigen have been immobilized; and 2) one or more subpopulations of fluorescently labeled beads to which RBD, N protein and S2 antigen have been immobilized is combined with patient sample, e.g., a biological sample from an individual suspected of having been exposed to SARS-CoV-2 virus. After incubation unbound sample is washed away. Biotinylated antibody specific for RBD, N protein and S2 antigen and biotinylated RBD, N protein and S2 antigen is added to the washed beads and allowed to incubate. The biotinylated antibody binds to RBD, N protein and S2 antigen bound to antibodies immobilized on each respective bead subpopulation and biotinylated RBD, N protein and S2 antigen binds to the antibodies bound to RBD, N protein and S2 antigen protein immobilized on the relevant subpopulation of beads. Unbound biotinylated reagents are then washed away and labeled streptavidin-PE (Phycoerythrin) is added to allow for detection of total bound immunoglobulin and bound SARS-CoV-2 antigen. The reaction is incubated and then washed prior to detection using a BioPlex 2200, Bioplex 200 or Luminex LX-200 platform. The identity of bead subpopulation is determined by the fluorescence signature of the dyed beads, and the amount of total antigen and antibody captured is determined by the fluorescence intensity of the bound labeled Streptavidin. The sample fluorescence intensity is compared to the fluorescence intensity of a set of standards or calibrators to generate a qualitative, semi-quantitative or quantitative result.

### Example 5

### Multiplex Detection of SARS-CoV-2 Antibody and IL-6

A population of fluorescently labeled beads comprising: 1) a subpopulation of fluorescently labeled beads to which antibodies specific to IL-6 have been immobilized; and 2) one or more subpopulations of fluorescently labeled beads to which RBD, N protein and S2 antigen have been immobilized is combined with patient sample, e.g., a biological sample from an individual suspected of having been exposed to SARS-CoV-2 virus. After incubation unbound sample is washed away. Biotinylated antibody specific for IL-6 and biotinylated RBD, N protein and S2 antigen is added to the washed beads and allowed to incubate. The biotinylated antibody binds to IL-6 bound to the beads on which antibodies specific to IL-6 have been immobilized and biotinylated RBD, N protein and S2 antigen binds to the antibodies bound to RBD, N protein and S2 antigen protein immobilized on the relevant subpopulation of beads. Unbound biotinylated reagents are then washed away and labeled streptavidin-PE (Phycoerythrin) is added to allow for detection of total bound immunoglobulin and bound SARS-CoV-2 antigen. The reaction is incubated and then washed prior to detection using a BioPlex 2200, Bioplex 200 or Luminex LX-200 platform. The identity of bead subpopulation is determined by the fluorescence signature of the dyed beads, and the amount and/or presence of IL-6 and SARS-CoV2 specific antibody captured is determined by the fluorescence intensity of the bound labeled Streptavidin. The sample fluorescence intensity is compared to the fluorescence intensity of a set of standards or calibrators to generate a qualitative, semi-quantitative or quantitative result.

### Example 6

### Multiplex Detection of SARS-CoV-2 Antigens and IL-6

A population of fluorescently labeled beads comprising: 1) one or more subpopulations of fluorescently labeled beads to which monoclonal and/or polyclonal antibodies that bind SARS-CoV-2 RBD, N protein and S2 antigen have been immobilized; and 2) a subpopulation of fluorescently labeled beads to which monoclonal and/or polyclonal antibodies specific for IL-6 have been immobilized is combined with patient sample, e.g., a biological sample from an individual suspected of having been exposed to SARS-CoV-2 virus. After incubation unbound sample is washed away. Biotinylated antibody specific for IL-6 and biotinylated RBD, N protein and S2 antigen is added to the washed beads and allowed to incubate. The biotinylated antibody binds to IL-6 bound to antibodies immobilized on its respective bead subpopulation and biotinylated RBD, N protein and S2 antigen binds to the antibodies bound to RBD, N protein and S2 antigen protein immobilized on the relevant subpopulation of beads. Unbound biotinylated reagents are then washed away and labeled streptavidin-PE (Phycoerythrin) is added to allow for detection of IL-6 and bound SARS-CoV-2 antigen. The reaction is incubated and then washed prior to detection using a BioPlex 2200, Bioplex 200 or Luminex LX-200 platform. The identity of bead subpopulation is determined by the fluorescence signature of the dyed beads, and the amount of total antigen and IL-6 captured is determined by the fluorescence intensity of the bound labeled Streptavidin. The sample fluorescence intensity is compared to the fluorescence intensity of a set of standards or calibrators to generate a qualitative, semi-quantitative or quantitative result.

### Example 7

A total of 583 samples comprising 483 hospital normal (sample from subjects undergoing routine checkups) and 100 samples from pregnant women were tested for anti SARS-COV2 IgG antibodies. All samples tested were acquired before November 2019. Antibodies specific to nucleocapsid (N) are identified as “Anti-NC IgG” in Table 4. The percentages provided in the Table 4 refer the percentage of samples that did not contain IgG specific for the SARS-Cov-2 antigens.

TABLE 4
BioPlex 2200 SARS-Cov-2 IgG Assay Specificity

| | | Anti-RBD | Anti-S1 | Anti-S2 | Anti-NC |
|---|---|---|---|---|---|
| Sample Population | N | IgG | IgG | IgG | IgG |
| Normal¹ | 483 | 99.8% | 100.0% | 99.8% | 100.0% |
| Pregnancy | 100 | 100.0% | 100.0% | 100.0% | 100.0% |

¹Subjects undergoing routine check ups

### Example 8

Analytical specificity of a serology test is a measure of its ability to identify specific antibodies while excluding other non-specific antibodies. Cross-reactivity refers to the ability of a ligand to support binding of antibodies other than those intended to be measured and cause false positive test results. 283 samples collected from 32 diseased groups were tested for cross reactivity RBD and S1 antigens. The list shows cross reactivity for only two analytes; RBD and S1.

TABLE 5
BioPlex 2200 SARS-Cov-2 IgG Assay: Cross Reactivity

| Cross reactants | N | RBD IgG | S1 IgG |
|---|---|---|---|
| SARS IgG | 5 | 0 | 0 |
| MERS IgG Pos | 5 | 0 | 0 |
| Influenza A | 14 | 0 | 0 |
| Influenza B | 9 | 0 | 0 |
| Common Cold | 9 | 0 | 0 |
| Flu Vaccinated | 7 | 0 | 0 |
| 2008 Flu Vaccine BioAssay | 36 | 0 | 0 |
| validation panel | | | |
| Haemophilus influenza Ab | 5 | 0 | 0 |
| Parainfluenza virus Ab Type I | 4 | 0 | 0 |
| Parainfluenza virus Ab Type III | 2 | 0 | 0 |
| Metapneumovirus (HMPV) IgG Pos | 5 | 0 | 0 |
| Rhinovirus IgG | 5 | 0 | 0 |
| Streptococcus Pneumonia | 1 | 0 | 0 |
| Immunity Control | | | |
| CMV IgG Pos | 1 | 0 | 0 |
| EBV VCA IgG Pos | 23 | 0 | 0 |
| Measles IgG Pos | 13 | 0 | 0 |
| MMRV IgG Pos | 1 | 0 | 0 |
| Mumps IgG Pos | 14 | 0 | 0 |
| Rubella IgG Pos | 6 | 0 | 0 |
| VZV IgG Pos | 2 | 0 | 0 |
| HCV Ab Pos | 14 | 0 | 0 |
| Hepatitis A IgG Pos | 20 | 0 | 0 |
| Hepatitis B Pos Ab | 21 | 0 | 0 |
| Toxo IgG Pos | 1 | 0 | 0 |
| Syphilis IgG Pos | 2 | 0 | 0 |
| Elevated IgG | 1 | 1 | 0 |
| RF | 47 | 0 | 0 |
| ANA Positive | 4 | 0 | 1 |
| dsDNA Pos | 2 | 1 | 0 |
| Jo-1 Pos | 1 | 0 | 0 |
| RNP Pos | 1 | 0 | 0 |
| Sm Pos | 2 | 0 | 0 |
| Overall | 283 | 2 | 1 |

### Example 9

578 unique samples from COVID patients were procured from commercial vendors. Of the 578 samples, 453 (78.4%) came with complete demographics and PCR test results. All samples were tested by the BioPlex 2200 serology IgG assay. Serology IgG data was collected for all four analytes (RBD, S1, S2 and N (referred to as nCapsid in Tables 6 and 7).

TABLE 6
Number of positive samples exhibiting IgG

| | | Number Positive Samples | | | |
|---|---|---|---|---|---|
| Days Post | | Anti- | Anti- | Anti- | Anti- |
| Symptom Onset | N | RBD IgG | S1 IgG | S2 IgG | nCapsid IgG |
| ≤7 | 78 | 59 | 57 | 41 | 59 |
| 8-14 | 67 | 55 | 53 | 32 | 58 |
| 15-30 | 124 | 112 | 109 | 51 | 108 |
| 31-60 | 226 | 212 | 209 | 102 | 208 |
| >60 | 83 | 75 | 74 | 45 | 74 |

TABLE 7
Number of positive samples exhibiting IgG (expressed as %)

| Days Post | | Number Positive Samples | | | |
|---|---|---|---|---|---|
| Symptom | | Anti- | Anti- | Anti- | |
| Onset | N | RBD IgG | S1 IgG | S2 IgG | Anti-nCapsid IgG |
| ≤7 | 78 | 76% | 73% | 53% | 76% |
| 8-14 | 67 | 82% | 79% | 48% | 87% |
| 15-30 | 124 | 90% | 88% | 41% | 87% |
| 31-60 | 226 | 94% | 92% | 45% | 92% |
| >60 | 83 | 90% | 89% | 54% | 89% |

### Example 10

### Detection of Anti-RBD and Anti-S1 IgG Antibodies in Patients Infected with Wild Type and SARS-CoV2 Variants

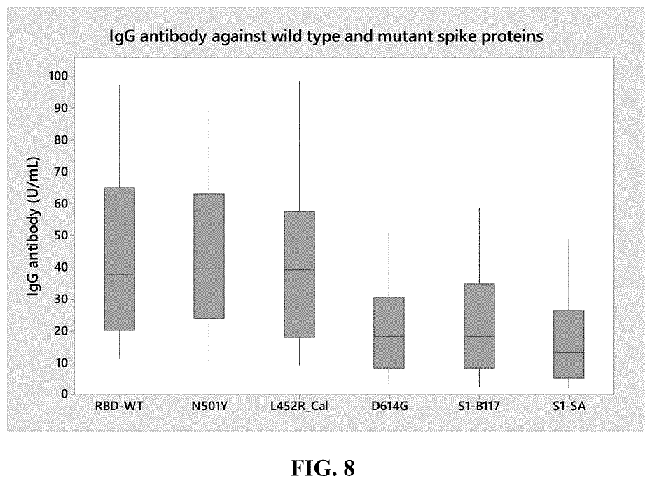

To address whether serology IgG assay can detect the variants of interest, recombinant RBD and S1 proteins representing variants of interest were evaluated for binding to sera from COVID-19 patients. Sera from 55 COVID-19 positive patients were tested for the presence of anti-RBD and anti-S1 antibodies against wild type and mutant proteins. RBD mutants used in this study include N501Y and California variant mutant L452R while S1 mutants are represented by D614G, UK mutant B1.1.7 and South African mutant S1. The box plot provided in FIG. 8 shows minimum, maximum and median IgG antibody levels (U/mL) as measured by their reactivity to recombinant proteins immobilized on the magnetic bead surfaces. Bead signals are calibrated independently for each protein and processed into individual antibody index (AI) values. Variant L452R represents California variant. Thus, FIG. 8 shows the reactivity of SARS-CoV2 specific IgG antibodies to wild type and mutant spike proteins and higher antibody reactivity was observed for wild type and mutant RBD proteins as compared to S1 mutant proteins. These observations support the detection of IgG antibodies against wild type and mutant proteins by the serology IgG assay.

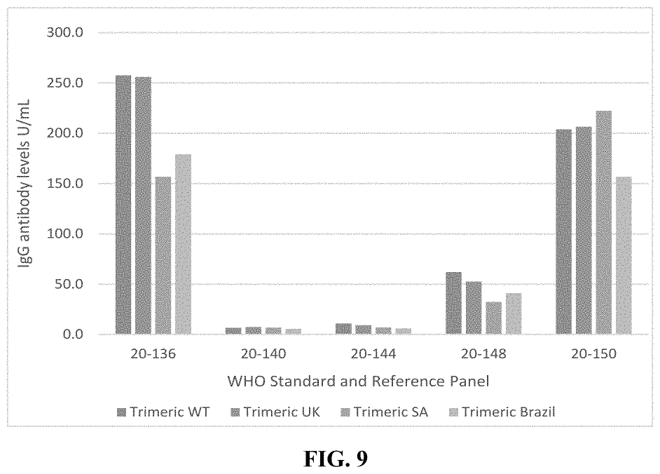

Further proof that a serology IgG assay can bind to trimeric spike proteins comprising S1 and S2 domains of the SARS-CoV2 virus from B.1.1.7 (UK), B.1.351 (South Africa) and P1 (Brazil) was obtained by analyzing the seroreactivity of WHO standards and a reference panel (see FIG. 9). Trimeric proteins from newly emerging SARS-CoV2 variants exhibit binding to a WHO patient panel sera supporting the utility of serology IgG assay in detecting COVID-19 antibodies in variants of concern (VOC).

### Example 11

### Detection of Antibodies in Patients Infected with SARS-CoV2 and Vaccinated Individuals

The ability of the serology IgG assay (with its ability to detect anti-RBD, anti-S1, anti-S2 and anti-nucleocapsid antibodies) to successfully discriminate natural infection from vaccinated subjects was examined by analyzing the antibody profile of infected and vaccinated subjects. A total of 195 PCR positive SARS-CoV2 patients and 66 vaccinated subjects were tested for the presence of anti-nucleocapsid (nucleoprotein) antibodies. The 195 COVID-19 patients ranged from 14 to 196 days post-onset of symptoms and the 66 Pfizer-BioNTech and Moderna mRNA vaccinated subjects ranged from 10 days to 62 days from the date of first immunization with vaccine. The subjects were evaluated for the presence of anti-RBD, anti-S1, anti-S2 and anti-nulear capsid antibodies and the results are provided in Table 8. As demonstrated by the data, a majority of infected patients demonstrated anti-nucleocapsid (nCapsid) antibodies, none of the subjects vaccinated with the Pfizer-BioNTech and Moderna mRNA vaccines demonstrated anti-nucleocapsid (nucleoprotein) antibodies.

TABLE 8

| Cohort | RBD WT | S1 WT | S2 | nCapsid |
|---|---|---|---|---|
| Infected Patients | 90.3% | 84.6% | 52.3% | 79.0% |
| Vaccinated Subjects | 98.5% | 98.5% | 78.8% | 0.0% |

### REFERENCES

1. Cheng Z J and Shan J. 2019 Novel coronavirus: where we are and what we know. Infection. 2020, Apr. 48(2): 155-163. doi: 10.1007/s15010-020-01401-y.

2. Cui J, Li F, and Shi Z L. Origin and evolution of pathogenic coronaviruses. Nature Reviews. 2020, Mar. 17: 181-192. doi: 10.1038/s41579-018-0118-9.

3. He F, Deng Y, Li W. Coronavirus disease 2019: What we know? Journal of Medical Virology. 2020, Mar. 14. doi: 10.1002/jmv.25766.

4. Guo L, Ren L, Yang S et al. Profiling Early Humoral Response to Diagnose Novel Coronavirus Disease (COVID-19). Clinical Infectious Disease. 2020, Mar. 21, pii: ciaa310. doi: 10.1093/cid/ciaa310.

5. Weiss S R and Navas-Martin S. Coronavirus Pathogenesis and the Emerging Pathogen Severe Acute Respiratory Syndrome Coronavirus. Microbiology and molecular biology reviews. 2005, Dec. 69 (4): 635-664. doi:10.1128/MMBR.69.4.635-664.2005.

6. Zhang W, Du R H, Li B, et al. Molecular and serological investigation of 2019-nCoV infected patients: implication of multiple shedding routes. Emerging Microbes & Infections. 2020, 9:1, 386-389. doi: 10.1080/22221751.2020.1729071.

7. Zhao J, Yuan Q, Wang H, et al. Antibody responses to SARS-CoV-2 in patients of novel coronavirus disease 2019. Clinical Infectious Disease. 2020, Mar. 28, pii: ciaa344. doi: 10.1093/cid/ciaa344 13 [EN].

8. Zhou P, Yang X L, Wang X G, et al. A pneumonia outbreak associated with a new coronavirus of probable bat origin. Nature. 2020, Mar. 579 (7798): 270-273. doi: 10.1038/s41586-020-2012-7.

9. Fan Wu, Aojie Wang, Mei Liu, et al. Neutralizing antibody responses to SARS-CoV-2 in a COVID-19 recovered patient cohort and their implications. 2020 Apr. 29 doi: https://doi.org/10.1101/2020.03.30.20047365.

10. Juanjuan Zhao, Quan Yuan, Haiyan Wang, et al. Antibody responses to SARS-CoV-2 in patients of novel coronavirus disease 2019. 2020 Mar. 3 doi: https://doi.org/10.1101/2020.03.02.20030189.

We claim:

1. A method for detecting SARS-CoV2 antibodies and, optionally, cytokines in a mammal comprising obtaining a biological sample from the mammal, contacting said biological sample with a substrate and detecting the presence or absence of antibodies bound to SARS-CoV-2 proteins or fragments thereof and, optionally, the presence or absence of cytokines, said substrate comprising a population four or more separate subpopulations of particles or beads selected from:
a) the spike protein receptor binding domain (RBD) immobilized on a first subpopulation of particles or beads having a first specific detectable physical parameter;
b) S1 immobilized on a second subpopulation of particles or beads having a second specific detectable physical parameter;
c) S2 immobilized on a third subpopulation of particles or beads having a third specific detectable physical parameter; and
d) nucleoprotein (N) immobilized on a fourth subpopulation of particles or beads having a fourth specific detectable physical parameter.

2. The method according to claim 1, wherein the mammal is a human, non-human primate, canine, or feline.

3. The method according to claim 1, wherein said detection comprises contacting the substrate contacted with said biological sample with anti-immunoglobulin (anti-Ig) antibodies and, optionally, cytokine specific antibodies, the anti-Ig antibodies and cytokine specific antibodies being detectably labeled with a fluorescent dye, fluorophore, luminescent agent, electron-dense reagent, or radioisotope.

4. The method according to claim 3, wherein said anti-Ig antibodies are detectably labeled with a fluorophore specific for each class of immunoglobulin and a fluorophore specific for each cytokine, when assayed, and the sample fluorescence intensity is compared to a set of standards or calibrators to generate a qualitative, semi-quantitative or quantitative result.

5. The method according to claim 1, wherein said detection comprises contacting the substrate contacted with said biological sample with anti-Ig antibodies and cytokine specific antibodies, when assayed, that are biotinylated and contacting said biotinylated antibodies with a biotin-binding ligand that is detectably labeled with a fluorophore and determining the fluorescence intensity for each subpopulation of beads.

6. The method according to claim 5, the sample fluorescence intensity is compared to a set of standards or calibrators to generate a qualitative, semi-quantitative or quantitative result of total Ig bound to said substrate.

7. The method according to claim 5, wherein said method assays for the presence and/or quantity of one or more of said cytokines.

8. A substrate comprising at least two SARS-CoV-2 proteins or fragments thereof selected from the group consisting of spike (S) and/or fragments thereof, S1 and/or fragments thereof, S2 and/or fragments thereof, envelope (E) and/or fragments thereof, nucleoprotein (N) and/or fragments thereof and/or membrane (M) protein and/or fragments thereof and combinations thereof, said proteins and/or fragments thereof being immobilized on said substrate.

9. The substrate according to claim 8, wherein said substrate is glass, plastic, polystyrene or nitrocellulose.

10. The substrate according to claim 8, wherein said substrate is a particle or a bead.

11. The substrate according to claim 10, wherein said substrate is a population of particles or beads comprising two or more separate subpopulations of particles or beads, each subpopulation of particles or beads being distinguishable by a specific detectable parameter and each subpopulation of beads comprising a distinct SARS-CoV-2 protein or fragment thereof immobilized thereon.

12. The substrate according to claim 11, wherein said population comprises two or more separate subpopulations of particles or beads selected from:
a) Spike (S) and/or fragments thereof immobilized on a first particle or bead having a first specific detectable physical parameter;
b) S1 and/or fragments thereof immobilized on a second particle or bead having a second specific detectable physical parameter;
c) S2 and/or fragments thereof immobilized on a third particle or bead having a third specific detectable physical parameter;
d) envelope (E) protein and/or fragments thereof immobilized on a fourth particle or bead having a fourth specific detectable physical parameter;
e) nucleoprotein (N) and/or fragments thereof immobilized on a fifth particle or bead having a fifth specific detectable physical parameter; and
f) membrane (M) protein and/or fragments thereof immobilized on a sixth particle or bead having a sixth specific detectable physical parameter.

13. The substrate according to claim 12, wherein said population of particles or beads comprises four separate subpopulations of particles or beads selected from: a) the spike protein receptor binding domain (RBD) immobilized on a first particle or bead having a first specific detectable physical parameter; b) S1 immobilized on a second particle or bead having a second specific detectable physical parameter; c) S2 immobilized on a third particle or bead having a third specific detectable physical parameter; and d) nucleoprotein (N) immobilized on a fourth particle or bead having a fourth specific detectable physical parameter.

14. The substrate according to claim 11, wherein the specific detectable physical parameter is a fluorescent dye, fluorophore, luminescent agent, electron-dense reagent, radioisotope or particle size.

15. The substrate according to claim 14, wherein the specific detectable parameter is a fluorophore.

16. A method for detecting antibodies in a mammal comprising obtaining a biological sample from the mammal, contacting said biological sample with a substrate comprising a substrate according to claim 8 and detecting the presence or absence of antibodies bound to SARS-CoV-2 proteins or fragments thereof immobilized on the surface of said substrate.

17. The method according to claim 16, wherein the mammal is a human, non-human primate, canine, or feline.

18. A method of differentiating mammals infected with SARS-CoV-2 from mammals vaccinated with a spike protein vaccine comprising obtaining a biological sample from the mammal, contacting said biological sample with a substrate and detecting the presence or absence of antibodies bound to SARS-CoV-2 nucleoprotein immobilized on a substrate, optionally, the presence or absence of cytokines,
said substrate comprising a population four or more separate subpopulations of particles or beads selected from:
a) the spike protein receptor binding domain (RBD) immobilized on a first particle or bead having a first specific detectable physical parameter;
b) S1 immobilized on a second particle or bead having a second specific detectable physical parameter;
c) S2 immobilized on a third particle or bead having a third specific detectable physical parameter; and
d) nucleoprotein (N) immobilized on a fourth particle or bead having a fourth specific detectable physical parameter.

19. The method according to claim 18, wherein the mammal is a human, non-human primate, canine, or feline.

20. The method according to claim 18, wherein said detection comprises contacting the substrate contacted with said biological sample with detectably labeled anti-immunoglobulin (anti-Ig) antibodies.

21. The method according to claim 20, wherein said method quantifies antibody amounts in the biological sample by measuring the amount of antibody captured by each subpopulation of beads by the intensity of the attached labeled anti-Ig on each subpopulation of beads.

22. The method according to claim 21, wherein the labelled anti-Ig is labeled with a fluorescent label, the identity of each subpopulation of particles or beads is determined by the fluorescence signature of the dyed beads, and the amount of antibody captured by the antigen is determined by the fluorescence intensity of the attached labeled anti-Ig.

23. The method according to claim 22, wherein the sample fluorescence intensity is compared to a set of standards or calibrators to generate a qualitative, semi-quantitative or quantitative result.

24. The method according to claim 18, wherein said detection comprises contacting the substrate contacted with said biological sample with anti-Ig antibodies that are biotinylated and contacting said biotinylated antibodies with a biotin-binding ligand that is detectably labeled with a fluorophore and determining the fluorescence intensity for each subpopulation of beads.

25. The method according to claim 24, the sample fluorescence intensity is compared to a set of standards or calibrators to generate a qualitative, semi-quantitative or quantitative result of total Ig bound to said substrate.

26. A method of differentiating mammals infected with SARS-CoV-2 from mammals vaccinated with a spike protein vaccine comprising obtaining a biological sample from the mammal, contacting said biological sample with a substrate and detecting the presence or absence of antibodies bound to SARS-CoV-2 nucleocapsid immobilized on a substrate, optionally, the presence or absence of cytokines,
wherein said substrate comprises a population four or more separate subpopulations of particles or beads selected from:
a) the spike protein receptor binding domain (RBD) or RBD variant immobilized on a first particle or bead having a first specific detectable physical parameter;
b) S1 or a variant thereof immobilized on a second particle or bead having a second specific detectable physical parameter;
c) S2 or a variant thereof immobilized on a third particle or bead having a third specific detectable physical parameter; and
d) nucleocapsid (N) immobilized on a fourth particle or bead having a fourth specific detectable physical parameter, provided that at least one subpopulation of particles or beads provides a RBD variant, S1 variant or S2 variant.

27. A substrate comprising at least two SARS-CoV-2 proteins or fragments thereof selected from the group consisting of spike protein (S) and/or fragments thereof, S1 and/or fragments thereof, RBD or a fragment thereof, S2 and/or fragments thereof, spike protein (S) and/or fragments thereof, S1 protein variants and/or fragments thereof, RBD variants or a fragment thereof, S2 protein variants and/or fragments thereof, envelope (E) and/or fragments thereof, nucleocapsid (N) and/or fragments thereof and/or membrane (M) protein and/or fragments thereof and combinations thereof, said proteins and/or fragments thereof being immobilized on said substrate, wherein:
said substrate is a population of particles or beads comprising two or more separate subpopulations of particles or beads, each subpopulation of particles or beads being distinguishable by a specific detectable parameter;
each subpopulation of particles or beads comprises a distinct SARS-CoV-2 protein or fragment thereof immobilized thereon; and
at least one subpopulation of particles or beads comprises a SARS-CoV-2 protein variant selected from the group consising of spike protein variants and/or fragments thereof, S1 protein variants and/or fragments thereof, RBD variants or a fragment thereof, S2 protein variants and/or fragments thereof.

28. A method for detecting antibodies in a mammal comprising obtaining a biological sample from the mammal, contacting said biological sample with a substrate comprising a substrate according to claim 27 and detecting the presence or absence of antibodies bound to SARS-CoV-2 proteins or fragments thereof immobilized on the surface of said substrate.
